SEQUENCE LISTING (19 sequences)

SEQ ID NO: 1 - protein, 253 aa, Homo sapiens
MARSLLLPLQILLLSLALETAGEEAQGDKIIDGAPCARGSHPWQVALLSGNQLHCGGVLVNERWVLTAAHCKMNEYTVHLGSDTLGDRRAQRIKASKSFRHPGYSTQTHVNDLMLVKLNSQARLSSMVKKVRLPSRCEPPGTTCTVSGWGTTTSPDVTFPSDLMCVDVKLISPQDCTKVYKDLLENSMLCAGIPDSKKNACNGDSGGPLVCRGTLQGLVSWGTFPCGQPNDPGVYTQVCKFTKWINDTMKKHR

SEQ ID NO: 2 - DNA, 969 bp, Homo sapiens
ggatttccgggctccatggcaagatcccttctcctgcccctgcagatcctactgctatccttagccttggaaactgcaggagaagaagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaacctcatgccctgttgataaaccaatcaaattggtaaagacctaaaaccaaaacaaataaagaaacacaaaaccctcaa

SEQ ID NO: 3 - DNA, 9909 bp, Homo sapiens
ggcatggtggtgcacgcctgtaatccagctactcaggactctgaggcaggagaatcacttgaacacgggggagtggaggttgcagtgagccgagatcgtgccattgcactccagcctgggtgacagagccagagtccatcaaaaaaaaaaaaaaataagaaagattcttctctcctctatgtgtccatgcagtctcatcatttagctaccacttgtaagtaggaacatgccatatctggttttctgttcctgctttagtttgtaagggtaatggcctccagctccattcacgtccctacaaaggacatgatcgtgttcttttttatggctacgtagtattcaattgtgtatacgtaccacattttcttaatccagtctatcactgatggacatttaggttgattccctgtgtttgctgttgtcaatagttctacaatgaacgtacgtgtccatgtgtctttaaacagaatgatttatattcctttgggtacacacactggggcttatgagagggtggagagtgggaggaaggagaggatcagaaaaaaataactaatgggtactaggcttaatacctgggtgattaaataatctgtataacaaacccccatggcgcacgttcacctacgcaacaaacctgcacatcctgcacatgtacccccgaactgaaaagttaaaaaaagaaaaataaatatttgcttataaattaataaatgaagccctcaaaaatgttctattagataatgttaagtacagacatttttgttataaatacataatatacaaagaaatctatgtataacatgattaaaatgaccataagaacatagatcctaaacatggcaaatattagtggggtggggttagggaaagcgttgtttttaacttacacctctctgttagagttgggaatgggttcaggcgtaattacaggcacgactgggatcagcttggacaagttcccccaggcgggccagaattaggatgtagggtctaggccacccctgagagggggtgagggcaagaaaatggccccagaagccgggcgcagtggctcacgcctgtaatcccagcactttgcggggccgaggcgggcacatcatgaggtcaggagatcgagaccattctggccaacatagtgaaacccggtctctactaaaaatacaaaaattagctgggagtggtggtgcgtgcctgtaatcccaggtactcgggaggctgaggcaggagaatcacttgaacctgggaggcggagctggcagtgagccgagatcgcgccaccgcactccagcctggcgatagagagagactccatccaaaaaaaagaaaggaagggagggagggaggagggaagaaagaaagaaaaccgccccagagaaggacccgagccagagcctattctctgagctcagcgactgcttgaatcccgctcctgcccctcagacccagcgcaccgggtccctcccccgagagcagccaggagggactgtgggaccagaatgtgcgggggcgcaggagctgggcaccgcccgtccttcggagggagggtggagagagagtgcagtggtgccaattgctctcgctgcgtcagggttccagataaccagaaccgcaaatgcaggcgggggtgtcccagagtcggctccgcctgcaccccagggcgctggggccgggcatggggcggggggtgatataagaggacggcccagcagagggatgaagattttggagcccagctgtgtgccagcccaagtcggaacttggatcacatcagatcctctcgaggtgagaagaggcttcatcaagggtgcacctgtaggggagagggtgatgctggctccaagcctgactctgctctcgagaggtaggggctgcagcctagactcccggtcctgagcagtgagggcctggaagtctgcaatttggggccttttagggaaaaacgaactacagagtcagaagtttgggttccacagggaagggcaagatcggagcctagattcctgggtctctagggatctgaagaacaggaattttgggtctgagggaggaggggctggggttctggactcctgggtctgagggaggagggcctgggggcctggactcctgggtctgagggaggaggggctgggggtctcgactcctgggtctgagggaggaggggctgggggcctggactcctgggtctgagggaggaggggctgggacctggactcctaggtctgagggaggaggagctggggcctggactcctgggtctgagggaggaggggctggggcctggactcctgggtctgagggaggaggggctggggcctggactcctgggtctgagggaggaggggctggggcctggactcctgggtctgagggaggaggggctggggcctggactcctgggtctgagggaggaggggctggggcctggactcctgggcctgagggaggagggactgagacctggactcctaggtctgagggaggagggactgggacctggactcctgggtctgagggaggaggagctgggggcctggactcctgggtctgagggaggaggggctggggcctggactcctgggtctgagggaggaggggttggggcctggactcctgagcctgagggaggagggacttggacctggactcctaggtctgagggaggaggagctgggggcctggactcctaggtctgagggaggcggggctgggggcctggactcctgggtctgagggaggaggggttggggcctggactcctgagcctgagggaggagggacttggacctggactcctaggtctgagggaggaggagctgggggcctggactcctaggtctgagggaggaggggctgggggcctggactcctgggtctgagggaggaaggtgctagggtctggactcttgggtatgagggaggaggaggttaggggtctggacttctgagtgtaaggaaggagaggccagagaaaggaatttctgggtctgagggaggaggggctggggttctggacccctaggtctgagggaggaggggctggggcctggacccctgggtctgagggaggaggggctggggccggtactcctgggtctgtggggggaggggctggggcctggacccctgggtctgagtggggaggggctgggcctgaatgctttctccttctcagctccagcaggagaggcccttcctcgcctggcagcccctgagcggctcagcagggcaccatggcaagatcccttctcctgcccctgcagatcttactgctatccttagccttggaaactgcaggagaagaaggtgaaagctggactgggaagtctgacctcacctcagggcccccactgaccctctccaaggagtccctgagtcagaacccttccctcctcaaacagcttccatcctgggaggaccagactgtcggctgaagcccccgctcttcctgcttctgctgactcagggggtctctgtcccctccaggccctgcctcctgtgctcagggtctctctgtggttccccagatgagatgcgcctcctgggtttctgagtgggctccttctgtctgtctctatccctatctcttgctttctctgtatttctccacacattttcatctgtctctgtccatctctgactctgggaatccctgaggtgcagcctcagccttcccctaatgctagctacccacgtgctcctccatgtctccatccagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaagtaggtgccgcccaagtctctgctggaggtgcaccagcgtctccagctcgctatgggggtggaagggcagtctttctgtgcctacggctctattctcctctctctgggtctctgtcctcctctctctgggcctctgtaccccctctccctggggctctgtccccctctctccctggctctctgtctccctctctctgggtctctgtccccctctctctggatctctgttcccctctctctgtgtctctgttccccattctctctaggtctctgttccccctcctctctctctgggtctctgtccctctctctctggatctctgtccccctctctctctgggtctctgttcccctctctctgggtctctgtcccctctcctctctctgtgtctctctcccccctcctctctctgtgtctctgtcccccctcctatctctgtgtctctctcccccctcctctctctgggtctctgtccccccctctctgggtctctgtctccctctctctggggctctgtccccctctctctctggatctctgttcccctctctctgggtctctgtctcccctcctctctctgtgtctctgtcccccctcctctctctgggtctctgtccccaccccgtcccccaggtctttgcacaccctctctgtcacagtgtctcttctgaatctgtgaatgtcactcctcgcagtgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatggtaggtggcctcagtgacccaggagtgcaggccccagccctcctccctcagacccaggagtccaggcccccagcccctcctccctcagacccaggagtccaggcctcagcccctcctccctcagacccaggagtccaggcccccagcccctcctccctcagacccgggagtccagaccccagcccctcctccctcagacccagcagtcctgggccccagaccctcctccctcggaaccaggagcctgaacaacagcccttctggtcctcgcccccatcctctctgactgacagctctccctgctcctccctgcagtgacctttccctctgacctcatgtgcgtggatgtcaagctcatctccccccaggactgcacgaaggtttacaaggacttactggaaaattccatgctgtgcgctggcatccccgactccaagaaaaacgcctgcaatgtgagaccctcccccccaattcctccccagtcctgggtaccctgtctgcatgccccagggacagagcttgacccgagtgactgggtaccaagcccggccttgccctccccccaggcctggcctcctcagctttttccacctcattctctgcctaggtcaggggtgggagtttacttaggggccaatgtggccctggggatgagacagagagtttaataggggtgagaaagtgggggtgggaccagggaaggagactgaggtgctggcctcaggcccaaaccttaagggggcaccaaaaacctcagtgattgagataaatcataatgcaatatttaaaaataaaaataaaaactcatgcagaagtccatgatggacaaaatgtcacattttaaataaagagcaggtggatcttactgaattttcccttgccgtaagtactagcgtggctcagcacagcgctgtactggcactgtcttcatttaaaatgtggataccatgcccatcatgcagttttatgtattacatttgatttcgttaagtactgcattgaagtattgtgtattgcagttactgagattttgtgcctgaagctgatgactcactcacctgaccctggccctggtcccggggaaaacactctttctctccacctcctctctgttccctctttctggccttttgtcatcccctctgtttctgaacagtcttcccacatctctctttgtgacataatttcatttcattcttttcctctttgttttttctctgtgttgagctagcttgctctccctcccttgttctctctccatgccctcctctctgctctctgtcttctccctctttctcttgcttctctctctctcctcccctccctctctcctctccctgcccccctgctctctcttttttcctctctctctgtctcctctctggccctctcctctttctctctctcccccacttctctgtctctcttcatctctctccctcatctctccttgccccctcctttttactgtctctctctttctctttcttctatctctctcctctccctgccgctcccccatctctgtctttctttctctctctttattctcctcctctcttccagtctctctctcctctccccacccccaccccatctctctccccacaccttccccccctttctctttgtctctctcttctccctctttcttctccacccccatctctctctctcttctcttcccacaccctccccatctccctcatctctttgtctgtctctcttctccctccttcttttccacccccatctctctgtctctctctctccccataccctttccctcttcctcatctctctttgtctctctctcctttccctctttcttctccacctccaactctctctgtctctccacacccatcctccttgctcacatctgcaccttcagctgtcaggggatgtgggatggtgagtgttagggatagaggagatgggagagagatgactgtcctagagaatagggtgttccccttctcccctggtgagggccagtttcatgaatgtgcaagctctgcacggacacagagccccacactcagaagggtctcaaacttagtctaatgcattcctgctgttgtcttgaaattctcaataatttttgaacaaagggccctgcattttcgttttgcaccaagtcctgtaaattatgtaactggtcttcaccctggtctccgagaccatcgtgtccccctttcctgcgccacagggcacgcatccaccccttggagatgatgttccttctcccactagcttggagcagggtccttaacattggaaaataaagagtgctctgatcctggaagccccaccccttctctgcaattggtctcattggccaagggtcaaaccagtgtcttcaaaggacctagtgtgtccctagcactagctctcccattagtccccagagacaatgagtctcttctcattggctatggtggaagtccataatctgcaagacaaagaccgataactgaggaatgtatgagaatgagttgggctttgatctgaagccaaagttaatctccggctctattccctctagggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaaccaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcatggtcttgaacgcctgacctcagatgatccacccaccttggcctcccaaagtgctgggattacaggcatgagccaccacggccagcccacaatgatattacaaacctattaaaaatgatacttagacagaattgtcagtattattcaagaacatttaggctataggatgttaaatgacaaaaggaaggacaaaaatatatatgtatgtgaccctacccataaaaaatgaaatattcacagaatcagatctgaaaacacatgtcccagactgcatactggggtcgtcatgaggtgtctccttccttctgtgtacttttccttgaatgtgcacttttataacatgaaaaataaaggtggggaaaaaagtctgaagatctaagattggagagaggtgacctttcaggaagggagactagaaagaaatatgtgcctggttttgagccctggtcctgccggccctgttccagggcatatttccatttcccagatctcagtttttcctgtctgtaaaatgggagagagaggaaaggatggagagaggaagaaggaagggaggagggaggagagaacaggccaacttcatcagcgtgggaaggggtgtgaaagtgtttctgagcatctcacgagtgacaagtgaggagggaggctggcggttttcagagggattgggatgacagtagacaggacacaggggtcccacgggggtctgccagaagtaagcaaacagtgccggaggaagatggtggcacctgctccccaagaagggagggaaaggaacctcgggaagcgggtaggatgagggaggagtcctctgtgactcagagcctggccacagccccagccatctaacatcaaagatcctctgtgtggtcacacctcagacgctgctgaccgaggagccactccagcccaggacacgccctcctacctgttcttcctgtttttctcccagaattccctccccaccaagatcctccagatccttcccctccttatctcatctccctctgagtctctcctaacccaggcaccacagccctgtcatattgcagaaattctgcagccgctaattctgattctcccatataggaggctaacacagaaaacgcaggagtccaggcccccagcccctccttcctcagacccaggagtccagaccccccgccccaacccctcctccctcagacccaggagcccaggtccccagccccttctgtttctgggcctgtcaagtttaagaatgtcaaacattttcgaccagtcattcccctgaagttttagcaacattttctctctcttctgcaaggcactccaacattcaatctggaattttaaaaagtaacaaaacattgcatttgcactaagtcagcctggagatccctggccctggccctctgctctcctatacgcaagctacaggtagattggtttgcaatgactgagatggtactaatgttgattttttttaagtaattcatttttctttgggtaagcagtatagtgtggtagttaagggactagctctggatcttggcttcttgggttcaaatcccagttctagtccctacaagctattttcctttaagctcattacttcccctgtccctgttccttcatccttgaaatgggagaaaaagcacctactttctagggttattacagagattcaataagttaatatacagaaagtgctcaaacattgt

SEQ ID NO: 4 - protein, 276 aa, Homo sapiens
MRAPHLHLSAASGARALAKLLPLLMAQLWAAEAALLPQNDTRLDPEAYGAPCARGSQPWQVSLFNGLSFHCAGVLVDQSWVLTAAHCGNKPLWARVGDDHLLLLQGEQLRRTTRSVVHPKYHQGSGPILPRRTDEHDLMLLKLARPVVPGPRVRALQLPYRCAQPGDQCQVAGWGTTAARRVKYNKGLTCSSITILSPKECEVFYPGVVTNNMICAGLDRGQDPCQSDSGGPLVCDETLQGILSWGVYPCGSAQHPAVYTQICKYMSWINKVIRSN

SEQ ID NO: 5 - DNA, 1580 bp, Homo sapiens
  Gene (1)..(1580)
  CDS (220)..(1050)
catcctgccacccctagccttgctggggacgtgaaccctctccccgcgcctgggaagccttcttggcaccgggacccggagaatccccacggaagccagttccaaaagggatgaaaagggggcgtttcgggcactgggagaagcctgtattccagggcccctcccagagcaggaatctgggacccaggagtgccagcctcacccacgcagatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggctccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgaccacctgctgcttcttcagggagagcagctccgccggaccactcgctctgttgtccatcccaagtaccaccagggctcaggccccatcctgccaaggcgaacggatgagcacgatctcatgttgctgaagctggccaggcccgtagtgctggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccctgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatggtgattgtcatgtaaggcttaacacagtgggtggtgagttctgactaaaggttacctgttgtcgtga

SEQ ID NO: 6 - protein, 276 aa, Homo sapiens
MRAPHLHLSAASGARALAKLLPLLMAQLWAAEAALLPQNDTRLDPEAYGSPCARGSQPWQVSLFNGLSFHCAGVLVDQSWVLTAAHCGNKPLWARVGDDHLLLLQGEQLRRTTRSVVHPKYHQGSGPILPRRTDEHDLMLLKLARPVVLGPRVRALQLPYRCAQPGDQCQVAGWGTTAARRVKYNKGLTCSSITILSPKECEVFYPGVVTNNMICAGLDRGQDPCQSDSGGPLVCDETLQGILSWGVYPCGSAQHPAVYTQICKYMSWINKVIRSN

SEQ ID NO: 7 - DNA, 5574 bp, Homo sapiens
  Gene (1)..(5574)
  gene (1)..(5574)
  promoter (1)..(47)
  mRNA (48)..(120)
  5′UTR (48)..(120)
  exon (48)..(120)
  mRNA (605)..(701)
  5′UTR (605)..(613)
  exon (605)..(701)
  CDS (614)..(701)
  mRNA (2455)..(2635)
  CDS (2455)..(2635)
  mRNA (3589)..(3863)
  CDS (3589)..(3863)
  mRNA (4195)..(4328)
  CDS (4195)..(4328)
  CDS (4793)..(4942)
  mRNA (4793)..(5474)
ttggggtcaaaagggaaggtcccgccaggggtccctgggcagaggataccagcggcagaccacaggcagggcagaggcacgtctgggtcccctccctccttcctatcggcgactcccaggtgaagctacctgcaccccacccgggttggggtggattgcgagaggatgggtgggaacccccgggccacaggcaggagccggcttagagcctcggtttctccactgcgggacgcggaagtccccccgctgtgaggttgagaaagaggctcccactggctccgagcctcggatccccaccccgcgcgtgaaggagggggaaacctcgggcgcggcgtggctgcagccagggtagctggggcgcggagagcgctccactcgggcacagggaggacgggaagatgccgcgaggggcgtcattagggtaattgtgcccattaccgtgttccagcccgaatctcccgtctgccagcccctggggttatcggctgcaggtcaaaagggtgcttgcgggctggggtggcccaggctgggcggatgagcgcggcgaggtgggggtctctaacggagcatctgttttaacccgcccctgcacacaccccagcagatcctggccatgagagctccgcacctccacctctccgccgcctctggcgcccgggctctggcgaagctgctgccgctgctgatggcgcaactctggggtaaggtgggggacagggggcggggagaggcgccggtgggaggcacgggcgggagggcaatgtgttcccgtcaccaagccccgcgcacctctcctcccccgagaccccagcacccacccagcgctccggagcacggcccgcccccaagtcagctgggcccttcttctggctcggcccctgggtgacccgccccactcaggccctgtccgatttctgccacccggatcctcgctcttccgtggactcttcggcgtgttctttctcctcctcttacgccgccccatcccggccccgctccattttacactaccgtgttttgttttgtttgtttgtttgtttgagacggagtctcgctctgtcgcccaggctggagtgcagtggcgcgatctcggctcactgcaagctccgcctcccgggttcacaccattctcctgcctcagcctcccgagtagctgggactacaggcgcccgccaccaagcccggctagttttttgtatttttagtagagatgggatttcaccgtggtctcgatctcctggcctcgtgatccgcctgcctcggcctcccaaagtgctgggattacaagcgtgagccaccgctcccggccacaccacagtgttttatcctgagtcttgccttaccgctttttgccctctcccctcactttttttcttcctctttccctttctctctcttttttctttttctttctttcttttctttctttcgttcttcttttccttccttcctttccttccttcctttcgtttgtttcttctttcttgttttactttctctcttattttttcttctttctttcttgttttttttctttctctctctttttctttccttctttttttttttttttttttgagacagggcagtgctctgtctccgtggctggagtacagtggcccaatcagagctcactgcagcctcgacctcctgggctcaagcgatactcagcctccagagtagctggtaccacaggcatgcaccaccacatccggctttttttttttttttttttttttttttttttgagacagggtctcactctgtcgcccagactggagtgcagtggcccaatctcggctcattgcaacctccacctcctgggctcaagcgatcctcccacctcagcctcccaggtagctgtgactacaggcgcatgcctccgcgactacttttttgttgttgttgttgtttgtttgtttttgtagagactgggtcttgctgtgttgcccgggctggtcttgaattcccgagctcaagcggtccaaccgcctcggcttcccaaagtgctgggattacaggcgtaaaccactgcgccccacccctctcctggttttcaatcccgttttgttattcacaccccttcctctccccgatccccgagttctatccccgcacccttacctccccgccgcgttcaatccccgcccctctatcgaccagcgacgttctagccagctctccaggcgcgctgcgttcagtccctgccctccagacccaccctattctgtctcattactccacgctaccctatcccagcttccttccactttcacgcgcttcttctcctcccattccttcggtgcacgcgaaacccccaatatttccctaccaccctcgcgttctggctgcgtcccccgtccccgaacgcagtccagtgccacagcccagctccaaccccaaacccagaccctgccctccgtgctttgattccgtcccctttctttctcccagccgcagaggcggcgctgctcccccaaaacgacacgcgcttggaccccgaagcctatggcgccccgtgcgcgcgcggctcgcagccctggcaggtctcgctcttcaacggcctctcgttccactgcgcgggtgtcctggtggaccagagttgggtgctgacggccgcgcactgcggaaacaagtaggaggagatccatccccgaggacgccacggggggctgtggaggcggcctccagggaggagcgggcagggcggggtctctcggaactcccacagctggaggtgcggtccccggtgtccttccagcaggaggagagaccgggcttgcgctgtggccgccaggggacggtgtggtccttttccgcatttctgggcccgtcactcctctcccgctgattctccttgagctctaggaggaggtggttgcctgcaacggatagaagccagggtccggtgtggtcaggaatttagaactaaaggaaaaggttcacttcgtgagtccccgttgaaggaggaagggttgggtattaccacagagaaaatgtggagttgggctgggctcggtggctcacgcctgtaatcccagcactttgggaggccaaggcgggtggatcacctgaggtcaggagttcaagaccagcctggccaacatggtgaaaccacttctcaactaaaaataaaataaaataaaaaataaaaaattttaaaaaaattaaaaaaaaaaaaaggctgggtgtggtggcgggtgcctgtaatcccagctactcaggaaggaggctgaggcaggagaactgcttgaacctgggaggtggaggttgcagtgagccgagattgcgccattgcattctagcctgggtgacaagagcgaaactctgtttcaacaaagaaaagaaaaagagaaaagaaaatgtggaaggcttacctaggtgtccaggcccccagccctcctcaattcctgcagatcctcagagctcaaacaactgattcctcctccccatgtccactgaggtccccttctcccacaaggccctcttccctcagactcttcctatctccaggccctgcttcactgcccacctgctttcccagtccctgtgaagggtttgccttcacatgcctcttccttcccccaggccactgtgggctcgagtaggggatgaccacctgctgcttcttcagggcgagcagctccgccggacgactcgctctgttgtccatcccaagtaccaccagggctcaggccccatcctgccaaggcgaacggatgagcacgatctcatgttgctaaagctggccaggcccgtagtgccggggccccgcgtccgggccctgcagcttccctaccgctgtgctcagcccggagaccagtgccaggttgctggctggggcaccacggccgcccggagaggcaagagctggggctctgaggccagaacctcaggaggagggggctgagggcctgaacccctgggtctgaggaaggatgggctggggactggattcctggatctgagggaggacgggctggggtcctagatgcctgggtctgtgagtctgaggggaggaggggctgggggcctggactcctgggtctaagtggggaggggctggggccaggattcttgagtctgaaggaggaggggctggggcttaggatagaaacggtcttgtatctggactcctggctccccaaggattgggggctggacccagggattactggcatattctcccttcagtgaagtacaacaagggcctgacctgctccagcatcactatcctgagccctaaagagtgtgaggtcttctaccctggcgtggtcaccaacaacatgatatgtgctggactggaccggggccaggacccttgccaggtagggtctgaacagggagagtctctgactcctgggagggaggacagggaggttatgggaaaagagcagaccctgtgcccgatcccaaactccattcccaaacccatccttgaccccaactcttacccagacctaaccccctcctcatccctatcctcaatcccatttccatcctaaccccaccccattcccatctccaagcccattttcatcccctcaccttccatgaactacaatcccaacccaagtctcactgtgccttcattctcatcccccagcccaacctcccataacctgaagtccacctccattcctaccttccagctcatacctaattccaaccccatcccatcctcgtctttatcccaacccaaccccttccttccccaccactgccccagatcccaaagtgacagctctcacgttggcacatttatttgatctctcctttctgccacccccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccccgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatgtgattgtcatgtaaggcttaacacagtgggtggtgagttctgactaaaggttacctgttgtcgtgatctgaccacgtcccggtgaaagcgtgtgtccagggaagaagtgcacagggtagcccccaatcccaaccttccatccccaacccttagggatgatggaaga

SEQ ID NO: 8 - protein, 276 aa, Homo sapiens; identical to SEQ ID NO: 4

SEQ ID NO: 9 - protein, 244 aa, Homo sapiens
MKKLMVVLSLIAAAWAEEQNKLVHGGPCDKTSHPYQAALYTSGHLLCGGVLIHPLWVLTAAHCKKPNLQVFLGKHNLRQRESSQEQSSVVRAVIHPDYDAASHDQDIMLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAK

SEQ ID NO: 10 - DNA, 1451 bp, Homo sapiens
  CDS (147)..(881)
gtcgacccacgcgtccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaattttattatgaaaaaaaaaaaaaaaaaaaaaaaaaaaagggcggccgc

SEQ ID NO: 11 - protein, 244 aa, Homo sapiens; identical to SEQ ID NO: 9

SEQ ID NO: 12 - DNA, 12483 bp, Homo sapiens
  mRNA (2001)..(2185)
  mRNA (3084)..(3135)
  mRNA (3559)..(3606)
  CDS (3567)..(3606)
  mRNA (4346)..(4502)
  CDS (4346)..(4502)
  mRNA (8122)..(8369)
  CDS (8122)..(8369)
  mRNA (9791)..(9927)
  CDS (9791)..(9927)
  mRNA (11805)..(12483)
  CDS (11805)..(11954)
acacttaaaaaatcttctgacttaaaaaaaaaagtatggtgattggaaaatgtaaatgtgcatgcgtgcttggcatcacatttcattggccaggacttccctggatgctaaaggtcctcaaatgccaggctggggggctgggacttggtcccaagggagatggggacccagggcacgtctgtgagaggaggggcaaggtcagcacaaggcacaggaaggtctctctggggcaagggatacagagaacagagggatcctggtccaggtgggagaggtgcagctctgagttggggttgagggtgtgggtacagagaggaagggaccccccagagagaggaggcagagggatagggcctggtcactgggttgtgcaacatcagacttgctgtctgtgaagatagcacgtcctgagaagaaggtgctgaggtcagtggggaccaaatgtgagagggagcacccggagagtatactgaataccgaagtagtcttcatccctggagtgatggggggtgcacaatgcaagatgacaattagattcaatgcaagacaaagaaaagggttggctgggaacagtggctcatgcctatggtcccagctcctgggaagactgaggcgggagggtcgcttgagcccaggagggttgaggctgccacgagcaaggatcgtgccactgcactccagcctaggcgacagaacaagaccttgtctcaaaagaaaaaagaacttttttttttaagttacctgtagtgcccagcccaagcaggtgctgagccagacttcattcctatcattgtccttattacgcagtgacttccccctcctcatttctctccactctgccacgcacacaccctcaccctccagcccataccaaccaccccaaccactgcctgtggtttcccatgtgcacccaggccaggcattttcacggcctttcctcctgacctacgcctggctcagctttctaggcccaagttcaaagacacctccctaaatcttcccagatccctctgctactgcccagcaccaccatcttatcacagccccacgtcgttcccaagtgctctccgatttctgcttaactccatgcctctcgctgtgtgtccgcatctcatcaataagtcctcaagtcctcttccatcctgctagcttcctcatcgctcgggaatcatccccgctacttcctggggaaactgactcccttctgggcacacacagtgctacccccggggaaatctaagaagagacccaggagaagataagcacggagagtcagagaatcaaggggaaagaaagggagagaggccgggcacagtggctcacacctgtaatccagcactttgggaggccaaggtgggtggatcacctgaggtcaggagtttgagaccagcctggccaacatggtgaaacctcttccctactaaaaatacaaaaaacatttagccgggcgtggtggtgggtgcctgtaatcccagctacttgggaagctgaggcaggagaactgcttgaactcaggaggcggaggttgcagtgaactgagatcacaccactgcactccagcctgagtgacagagcaagactccgtcaaaaaaaaagaaagaaagaaagaaaagaaggaaggaaagaaagaaggaaggaaggaagggaggaagggagagaggaagggagagaggaagggagagagagaaaaaaagagggagagagacacaaatacagagactgagatgggagagagagagagatggaagctccctcccctccatggccagggagacagatggagcaagagacctcaggggtgggcagacttggaggagaaggaccaggaggatgtggagtgccgaaatctccagtcagggccaggtgggcagtcagagactgcaaaggaggactgtcagacagggacaaaaggaagccattgatgtaaccgccctcccgcctgcccgccggaagagaggttgaggccggagctgctgggagcatggcactggggtgctgggaggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcaggtgtgtgagtcaccccaaccgcactgaacctgggcaggctgcttcccagtgccggagggctctagagcccggagtgagggcctgcaggtccctgggtggcacagagagtgctgggggtgcagggaggcctggggcaccatctgcttgccccagaggccggaatttgtcttcagacactttctttctccaaaacccggaggtctaaggactgagccgactagaacttcctctgcctcagattcaggccccagcccctcctccctcagacccaggagtttaggtcctagcccctcctccctcagacccaggagtccaagttcccacctcctccctcagactcaggagtccaggcccccagcccctcctccctcagacccaggagtccaagttctcacctcctccctcagacccaggagtccaggccccaagcccctcctccctcagacgcaagggtccaggcccccagcccctcctccctcagactcaggagtccaggcccccaagcccctcctccctcagacccaggagtccaggccctcactgcactcagggaccagtgctcccttccctggaggcctggtcaggggtcaccaagagcagagcgtgggggcgggaggaatgtgtgtgggaggcctgggtaaggaggaaaagggtgtagccagtctcctggctcagggacctgagagacaggggttaaaaggacgttccagaagcatctggggacagaaccagcctcttccagggaggcctgggagctgggggtgtgtgtctggcagtccctgcagccctgggctctgcggcccctgcgtcctccgcttggctctgccactgcatctgagtgtcttctctcctcacggctccccgcatttctaactctttctgcctcctcgtctcaaagctgttccttcccccgactcaagaatccccggaggcccggaggcctgcagcaggtgagatcacagacatcacagaacctgccgggtgggcggggtgggtggccattgcgcacagagccaggctccgaggaaaactcccatacagaggaagaacgctagggccccctatggtaaccctctcctgtcgacaggaaggcaaatcagtgcccaagaaagtagaaagatctaatcagaatctcaccatgggttactggaccagtggacgtagttgaattctctttggcactgttttcgtggatcctcttggaagatgtgggctgaggaagaataaatcaggaggctagatgggaaggacagaggtcaaggcaggagaccatagcaggccaggaaggaaggagaggatgcagagggagcagacagagggatggggggagggtcgaggcagtgactaatggaccatgtggcttcccctctcaggagcggccatgaagaagctgatggtggtgctgagtctgattgctgcaggtggggaaagggcatttggatgggggaggcttgcagacagggttgggcttgttgatggagaagaggctggtattggggatggggatatgcacagggttggggtgggggagctttgaaatgaggaagacgttggggattaggctaagggtggggaatacagatagggagggtggtgggaggtgggtttgaagatatgagggtttggggtggggttggctttagggatggggatctaaacatagaagaggtaggaggtaggttggaaagttggagagagcccgggaataggggatacagttgggtttgtaatgggaatggggtaagtttgggagtggaaatacagagaagctttttttttttgagacagggtctcactctgtcacccaggctggagtgtagtggcatgatccatagttcactgcagacttgaactcttgggtctcaagtgaccctcccacctcagcctcccaagtagctgggactacaggcgtatgccaccataccctgctaatttgtgtgtgtgtgtgtgtgtgtgtgtgtgtgtgtgtgtgtggagatgaggtctcactgtgttaccgaggctggtctcaaactcctgggctcaagcgatcctcctgcctcagctgggattacaggcataagccactgcacctgaccaatcttgactggagttcatgttgagggggatgcgcttggtttctccagaactcctctctgactcagatcttctctccctcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgtgagtctacactgtaaatgaacagcagatgcgactgaaccctgagggtgtcttatagatgtcaggcaggaggtgacataggcatcccccccatcccagcacgaggccatctgatagccaggtgcattcggctgttgcttaattgagtacttaatgtgtgccaggccctgcgggcatagcagtggaaaagaaaataaaaaaaagaaaacaaaaaaaaacaagcaaaattgctgttttcctgaacttactttctaatgggggaattggatcatttggggacctgcagggcgtgatgggcatttggatttaattctgagcacagtaggaagccactgggcagttttgtttttgttgtttgtttgttttttgagacacagtctcgctctgtcacccaggctggagtgtagtggcatgatctcagctcactgcaacctctgcctcccaggttccagcgattctcctgcctcagcaccccaagtagctgagattacaggtgtgcaccaccttgcctggctaatttttgtatgtttggtagagacggggtttcaccatgttggccaggctggtctcgaactcctgacctcaggtgatccgcccgcctcgccctcccaaagagctgggattacaggcatgagccaccaccacacccagcctgatttacatttttacaagcaccctggctaccacgtggaacgtggtctgggcaagagagagggagggaggcccacgtgggggctgttgctttcatccggcgacataggagggtggcttgaacccaggcggtcgcagtggggatggagggatgttgaatatcttgggatgtggaattctgagactgagccagcagaatctggcaacgaggaacaggagggagaggaagaagcacggctggcttccgtgtatttgtcctgaacaactgggtgttttgccacgtctttctctgagttgtgggagagggaaagagaaacaggccgggtgtaggcaggggagcatctgacattttgctttagccacgatgagttggagatgccggggagatgtcccagcagggaggccagggaggactctggagctcagaggagaggtcagggctggaggttaaaatgaaggcatcgtcagcaaacaggtgtatttaaagccatgggactagatgagatcatccaaaaagctggcatagttggaggagctggagggcccaggacaaaaaccctgggcgctgatcctcactagtcagattcacgacagctgccacttgtttgatgctaactaccaatcaggtgctgagtgaaaccatgtacacacctttcctggaatgcccaccacaagggactcttggcaccattttgcaaatgaggaaactgaggtgcagggaaatagcaagtgacaatccctggggtggttcccctgaccccaaggagaccttggatgactctcaccaccatcattcattcctttgatgtacattgactaagagcacctgctaagtgccacattcgagttgggcagtggagattcagcaatggatgggacacacacgtcatccctgccctcgggagcacaaggacagaaaggtgcagacaagcaaagtgagggctgggcatggtggctcacgcctgtaatcccagcactttgggaggccgaggtgggtggattacctgagttcgagaccagcttggccaacatggctcaaccctgtctctactgaaaatacaaaaaattagccaggcgtggtggtgggcttctgtaattccagcaacttgggaggctaaggcaggagaattgcttgaacgtgggaggcggaggttgcagtgagccgagatcgcgccactgcactccagcctgaaccacagagcgagactctgtctaaaaaaaaaaaaaggaaagaaagaagcagcaaattgggctggccgtggtggctcatgcctgtaatcccagcactttgggaggccgaggcgggtggatcactcgagcccaggagtacaaagctgcagtgagctgtgatctacagaacaccactgcagatccagcctgggtgacagagcgagaccctgtctcaaaaaaacaaacaaacaaaagaagcaaacccttcaaaaccccatataattacaaattatgaaggaaaagaatacgggtacctactttagatggaggagggtcaggaaggactttctaatgagataaaatccaagcggaggcatgaagatgggaaaaggaatgttcagggcagaggaaaggctgtgataacacccctgaggtgagaaccgtcttgagtattctcagaaaataaaatttcccgttcactggggggcagaaggtgctgggagataaggttggaaagtgactacagccagatcacacaggggctccagtgccaagtggaggagcccaggctttattcttaggacaatggggagccatgggtgatgtctgagcaagggagtgactctctgtttcaggaatatgtatcaaacacctatcctgtgccaggtgctgatcaacgcactggagatactatatctgaatagaacaaaaatccccatcttgacatcctagagctgcactgtctaatatggtagccatcagccacatatagcaaattacattgaaattaatgaaatggaaaatccacaagccacatttcaagtactcagcagccacctgtagcttgtggttcccccagccacctctggacagtgcagatcgagatcatggcatcgtagcatttagtggacagcattgctctgcaaggaggagaaataacacaatgagtaaatatttaacaataaatatatagcaggtcggatgattgtgataggttctctggtggaacagaaagcaggggagggagataggaattgcctactaacaggtatttgtattttaattgggcaactaaggaaggcttccctgagaggcgacatttaaaggaagtgagggagtgagctatgcagatacttggaggacagacttgctggcagagggaacagcagtgcaaaggccctgaggtgggaagatcactattgtgttcaaggcaagacagggaagccagcgtttggctggagcagagggagagaaggggagagtgggaggagaagatgtctgtgagatgatggggcagtgcttgcaaggcctggtgtgccacgttgagaactttggctttgattctgagtgagatgggagtcataggaggggctgagcagaggaggcacaggaccaacttacattgttaaaatatctctggttgctttgtggaggatggactgtgggggaccagagacagagcagggagcccagtgaggaggctactgctctagttcaggtaggaagtgaaaaggcagctcaaaccaagatggtagccgtgggaaaggtgagatgtggccagattctggatatgcttcagagaggcaaaaggaattctggacagcttggatgtagggcatgaaataaagagagtgaagaatagcccccaagattattctgaaaggatggaattgccatttacccagctggggaagactgtgggaggagcaggccagcgattcatgacttcccagccctctctgaagcctcaactgcagcccaagggctccaggtgagacccagccctcttccttcccaggaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtcagtagtgggaggctggtggggagcaggctactggctacttggggaagtgtgccaaaggatggggagtgggaaaattggtgaggggccatgggaagatgggctaatggtgaggaccaatgggacagggtttcaatgggagaaaggtcaagggggagggagagtgaatttgggagctgggccagtgagtgaacagccaatggaaaatgtagaccaatgggtgaatagcatgggagagatggaacataagatgaaggttcaataaagagggaaggtcagtggggagatgctaatcaggaaggatgtcaaaggtcaaaggggactgatcaggattcattgaacagcaggaaggaataatggagaaggaactgatggaagaagagaaaccaataaagcacaaaagccaactgaaggatgtgaattgagacagtgaatgggggtatagctgatggaagagggactaaggggaaaggatcaatggtccagaggagtcactagaggaaaaaacaggtccaatagatcagcaggatccatgaaggtgggcctgtgtgtgaagggccaataagaaaggtgaaccattggatgaagggccagtgggaaggcagagacaatgggggaggatgcggcaagttagaaaaggaccaatgagggaggtggaccattggatgaagggctaataggaagggagagccagtgggggatggtgaggccagttagaaaaggaccaaggagggaagcagaccaataggaagagagagccaatgagggagggcagggccagttaggaaaggaccaatgaggaaggtagaccattggaggaagggccaatagaaagggaggatccatgagggagggtggggacagttagaaaaggaccaatgatggaggtggaccattggatgaagaaccaatagaaaggaagaaccaatgggagagggcatggccagttaggaaaagaccaatggtcacagagtgaccaatcaagatgaatcaatgggcaggaagtgtccaatgaagaatggactactgatcaggaggggtacagtagaggagggcgtaacagaggaagagtcctccaggtcaactgaaactactgaagaaggtgggaccagtggaagagagaaaagtggaggagggacctaagagaaaaggaaaaccaataggaaatgaggactcctggagaagagactattaatgaggaagacagccaatgggggggaagaatgatagaaagagggaccaattaggaggcagggacgatggtaatgagatgtaagaatgagagacaaacaggaagaggggtgccaatagaaaagagggaccaatagaggatggaggacttataggggttggggggtgactggggaggatgaggggagtgcaaggcctgggctgagtctggcccatctctcccctaacaggtgatttccctgacaccatccagtgtgcatacatccacctggtgtcccgtgaggagtgtgagcatgcctaccctggccagatcacccagaacatgttgtgtgctggggatgagaagtacgggaaggattcctgccaggtgaggtgacccggatctgccacttacacagccagggacaggacgaagtcacaaaaacatggccagacacaggaagagagagacacaggccaaaagagagctttacagagacagatagagacaggctgagggagaacccaagccttgaaaagaagagacttagttcaacacacagagacacagtcagggatatgcagagatataaagacacagccagcagagacaggaagtgcagagacaaggatggaggccgcgggatcaagaaccagagaggccaggagcagcggctcatgcctgtaatcccggcactttgggaggccgaagcaggaggatcacctagggtcaggagttcgagaccagcctgatcaacatggtgaaaccctatctctactaaaaatacaaaaattaggatgggcacagtggctcatgcctgtaatcccagcaccttgggaggccgaagcaggaggatcacctggggtcaggagttcgagaccagcctgatcaacatggtgaaaccctatctctactaaaaatacaaaaattaggatgggcacagtggctcatgcctgtaatcccagcaccttgggaggccgaagcaggaggatcacctggggtcaagagattgagaccagcctggccgatatggtgaaaccctatctctactaaaaatacaaaaattagctgggcctggtgcaggcgcctgtagtcccagctactcaggaggctgtggcaggagaatcacttgaacctggaggcggaggttgttgcagtgagtcgagatcatgctactgcactccagcctggcaacagagcaagattccgtctcaaaaaaaaaccaaaaaacaaaaattacgcaagcatggtgggacacacctgtagtcccagctactcgggaggctgaggctggagaattgcttaaacccaggaggcagaggctgcagtgagctgagatcacgccactgcactccagcctggggacagagccagactctgtctaaaaacaaaaagaaccaaagagaagtagtaaggaagcagatggtgtgaggggactgtccttcctcaaacagagcccccacgagtcctgctcagaaacgaccaggctctggaggagggagacactagctggggaaaggggactccctcccgaatactttaacttgggtttcctccattgtcatccatccaggctctcctctttatgccagaatgactaatgcactgagggatgtgcagagaccaaccaagggggagacacaggcagaaacggagacacaggcagaaacagggacagagacagggaaagcgatacatagcaagttggacgcaaagaaagggcaggtgggcgagactgtcctcaagacacgaggtggagaggtgtccctggacagaatagtgccaggcatatctctccctgggccctccctacctctcccacctgggtcttatcgtctcctcctccccctcctccctctcctcctcttcctcctcctcctcctccctcatcatcttcttcttttctctctctctccatcggtctctacacctctgcctctctccacacctctcagtctccattcttaaattgtttctctttcttgctctctatgttcctctgcatcttggcattcctatctctgtgtctttgagtctcctttattctctctctaccattctctctctgtgcctttgtgtgtcttactgtctctctctctgtctctctgtccctgagtctttctctccatctttcagtaagtacctctgtccctttctacctctctctctgtcacacacacacacacacacacacacacacacacacacacacacacagtctctgggtttctatctgtatctgactttctccctctttcctgcagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaatttt

SEQ ID NO: 13 - protein, 244 aa, Homo sapiens; identical to SEQ ID NO: 9

SEQ ID NO: 14 - DNA, 1506 bp, Homo sapiens
  CDS (246)..(980)
aggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccatcaggcagtggctgttggaaagatttaagatttcacacctatgacatacatgggatagcacctgggccgccatgcactcaataaagaatgtatttt

SEQ ID NO: 15 - protein, 244 aa, Homo sapiens; identical to SEQ ID NO: 9

SEQ ID NO: 16 - protein, 282 aa, Homo sapiens
MQRLRWLRDWKSSGRGLTAAKEPGARSSPLQAMRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 17 - protein, 250 aa, Homo sapiens
MRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 18 - DNA, 1186 bp, Homo sapiens
aggaatctgcgctcgggttccgcagatgcagaggttgaggtggctgcgggactggaagtcatcgggcagaggtctcacagcagccaaggaacctggggcccgctcctcccccctccaggccatgaggattctgcagttaatcctgcttgctctggcaacagggcttgtagggggagagaccaggatcatcaaggggttcgagtgcaagcctcactcccagccctggcaggcagccctgttcgagaagacgcggctactctgtggggcgacgctcatcgcccccagatggctcctgacagcagcccactgcctcaagccccgctacatagttcacctggggcagcacaacctccagaaggaggagggctgtgagcagacccggacagccactgagtccttcccccaccccggcttcaacaacagcctccccaacaaagaccaccgcaatgacatcatgctggtgaagatggcatcgccagtctccatcacctgggctgtgcgacccctcaccctctcctcacgctgtgtcactgctggcaccagctgcctcatttccggctggggcagcacgtccagcccccagttacgcctgcctcacaccttgcgatgcgccaacatcaccatcattgagcaccagaagtgtgagaacgcctaccccggcaacatcacagacaccatggtgtgtgccagcgtgcaggaagggggcaaggactcctgccagggtgactccgggggccctctggtctgtaaccagtctcttcaaggcattatctcctggggccaggatccgtgtgcgatcacccgaaagcctggtgtctacacgaaagtctgcaaatatgtggactggatccaggagacgatgaagaacaattagactggacccacccaccacagcccatcaccctccatttccacttggtgtttggttcctgttcactctgttaataagaaaccctaagccaagaccctctgcgaacattctttgggcctcctggactacaggagatgctgtcacttaataatcaacctggggttcgaaatcagtgagacctggattcaaattctgccttgaaatattgtgactctgggaatgacaacacctggtttgttctctgttgtatccccagccccaaagacagctcctggccatatatcaaggtttcaataaatatttgctaaatgagtg

SEQ ID NO: 19 - DNA, 9120 bp, Homo sapiens
tgataatagtgttctctctcctcattggtcagggccccagccattgtccttgagagaatgctcgactctttatgttgtcttgacagcctcccctgagattggtcattaatgactgtgctctctctcctcattggtcagggccccagccattgtccttgagagaacctctgtcctttatggagttccacccttcttccctgggattggcccctagagacagtggttcttctcttttggttagccattgccattgtcctccgggaaagtgattatactcttttgtctaatgaccagacttggagccctccccaaggcccaggactgggttgaagggttggggaggaaaacagaaataagatgtctcccttgttcagacagtacttctcttcccttccagggtgattctgggggccccctggtgtgtgggggagtccttcaaggtctggtgtcctgggggtctgtggggccctgtggacaagatggcatccctggagtctacacctatatttgcaagtatgtggactggatccggatgatcatgaggaacaactgacctgtttcctccacctccacccccaccccttaacttgggtacccctctggccctcagagcaccaatatctcctccatcacttcccctagctccactcttgttggcctgggaacttcttggaactttaactcctgccagcccttctaagacccacgagcggggtgagagaagtgtgcaatagtctggaataaatataaatgaaggaggggccatgtctgtccatttgaagtcctcatgctggttgagactggaagaaggactcagcagtttccctatctcataggagtagaaacagagctcaaataaggccaggcacagtggctcacacctgtaatcccatcactttgggaagctgaggcaggtggatcacctgaggtcaggaactcgggaccagcctggtcaacatagtgaaaccccaactctactaaaaatgcaaaaattagccaggcatggtggcgcatgcctgtaatcccagctactcaggaggctgagacaggagaatagcatgaacccgtgaggcagaggctgcagcgagccgagattgaaccattacactccagcctgggcgacagagcgagactccatctcaaaaacaaacaaacaaaaaacccagtgctcaaataggatgagggtcttccctgagtagttactcagaaatggagtagaaaaagttacttttaataatataggccgggtgcagtggcccacgcctgtaatcccagcactttgggaggccgaggtgggaggatggcttgagctcagatttcgagatcagcctggcaacacagtgaaatcttgtcactacaaaaacacaaaaaattagctgggtgtggtggtgcgtgcctgtagtcccagctacttgggaagctgaggtgggaggatcacccgagccggggaggtggaggctgcaaagagccgagatcatgccactgcactccagcctgggcaataaagtgagaccttgtctcaaaaacaaaaacccagcaatataaataagacacatgtttcttcatctggcataatagaaatagtgcccagagcttataagcttttcaagagtccacaaaagacccgaaaaagaaaaagaaaattgttagctccaaaataccagatgaaagctgcaaagtcaacatttatgaccatttaatccaatgtccataaaacgtagcattctttccactagccaactgcagtttactttcttgtaatgaagcatacattgtatctttaatgtgggacgtggctttgttctaataagacgaagggtggagtgcaggcttggaaagcaggagagctcagcctacgtctttaatcctcctgcccaccccttggattctgtctccactgggactcaagaggtgaggagagaccatctccccaaatgcactgaagggaaactggaggagggagggagtgaggggtgatcataccagcggaggcacatttgctgagcccccccgcagtctgctctttccaagtggaccctcctggaagcctgatcccaacctcccctgcaagcaggtctgtcacccccatctctcagatgaagaaactgagccttgcaggggtggagtcccttgtccccacgtcataagggtagtcatagtagtaggaagaggaagcacctaggtttgaggccagggctggctgctgtcagaacctaggccctcccctgccttgctccacacctggtcaggggagagaggggaggaaagccaagggaagggacctaactgaaaacaaacaagctgggagaagcaggaatctgcgctcgggttccgcagatgcagaggttgaggtggctgcgggactggaagtcatcgggcagaggtctcacagcagccagtaagtgaacagctggactcgggctgcctgggcggcagggagaagcgggcaggggaagggtcagcagaggagcgaggccccagaggagccctggggtggagcacagccaagggctctgttccctttcctggactcggcttccacaggccctgacctgcctcccccaccctccggtcctgcccctgtgcctggcagcagccccacctgtgtgacatcccagcacaccccccctctccttgcaaaggagaagggagcggcctaggggaggccaggggcccacctgggctggggctgtggagagggagtggctgggacgggaggaaaaagagagacggagattagatggaagaagagggatttcaagacaaattgccagagatgcagtcagagagactgactgagagacacaaagatagaaggaattagagaaagggccacacagagccagacagagagagaagagtggagatggagacagggacgaggacagagaaaggcagacagacacatagggacagaaagagaaaaatcacacaaagtcagaattactgaatgacagggaatgacacatagaacgagacacagattcagagactcagggcagggaaaggaaggctgcagacagacagacagacagagggaggctgagacacagggagaagaggggcttggagaggtggcacaggcaggcagccagtgcctcagaggcctccggggagggccctcacacacaccccgccccggggcattaaggcagggcttggaggccagtcatcctgggcccgcccagggccgcccccctgccagcccgcctgcctggtgcctggcacctggcgctccaacccagcctacctgctgtagctgccgccactgccgtctccgccgccactgggcccccagagccccagccccagagcctgtgagtccaggaggaaagggaagctgcccctccccgtccaggtgtcagccctccccaaggacacctgtcccactcgggcacccatttctccctctgctctgtccttctctgcttgggtgggggttcctggcctctctctacacctctcacctccgatggctgtccgcagcctcagttacctctaatctccatggcttcagctgctgagctggccctctgctcccacccccgctggccagggcagcggagggcactggccctcccctcgaccagccccgcccagctttgcttggctgtccttcaaaagggcaggggtttggcggacagggcttcagcaagccgggtgatgggggtcccagacattgtctggggctgagccccctactcccctccagcagacctcaaaggctccatatcgctctgctgcgaagacaatgaaaaaggggtggctacggaacggtgtctggttccccttgtccttccaccccaagctgctggggcctggccagctctcaaggcaagaaggaaaacatcctctgacatgtgccggggaggtcccatggctgacttgaacagggccgaaccatggcttgacagctcaaagcccctcccaacgacttccacatggttcttggtatctcggaagcttctagctgtgaccaggccctctccaaggccaccctagacacctaagatatattttaagtgtttggagatctgagtgctgtgagaaacaggggatttccccaaccttgtttctcccaagtggggagcgggagcaggtgagggagagaggagagggcatgagccagcccccccctcccgatttccccgtaaagtgatgcggccccatgtccctccttgttcccagaggaacctggggcccgctcctcccccctccaggccatgaggattctgcagttaatcctgcttgctctggcaacaggtacgcaggggatgggggcagggcaggatcctccctcttgaatctctgggatcccctaaccctctgtgtctggacagtgacagggctgattccaaattacagaacaacccataaggcacctgaactggagcagtggtcatgagggcctggatgcccttctagataatccctttaaatgccaaaggaggagaggtcaagggggtcgtaaagggtcccgtggaggggctgaggaagctggagttgggggagcagtcactcaaagcgcccaggacaggggctactgaccaaccagtatggaagtatttccttttttttttttcccagagacaaagtcttgctctattgtccaggctggagtgccgtggtgccaacacggctcactgcagtcttgacttcccgggcttaagtgatccttaagccatctcagcttccccggtagctgggaccacaggcacctgccaccaagccaggctaattgtttaattgtttgtagagatgggggaggaggtctcactatgtttgcctaggctgatctagaactcctgggctcaagtaatcctcccaccttagcctctcaaagtgctgggattacaggcatgagccactgcatttgaccttatggaagtattttcatcctttaatacccgaccccagcatccagggcaacccagagggacaccagaccagggcccagaccacccactctctttctctcctccccacccccatttctgggagtcctcctggtctaccacctctccttcctgagccccttcttttgctctcaccccctccagggcttgtagggggagagaccaggatcatcaaggggttcgagtgcaagcctcactcccagccctggcaggcagccctgttcgagaagacgcggctactctgtggggcgacgctcatcgcccccagatggctcctgacagcagcccactgcctcaagccgtgggtgcgggggctggggcggtgccggggtggggggctgggaatggggagatggatggagagaagctcagggataggggtgctggtaaggggattagagatggggatgggtagtgtcagcaaggttgatgggctcgagttggtattgaaggtggggggatgaatggggttgggatggggctatggctgggaagggggcttcggtgggagacgtggaagaggttggaagcagagcgatgtttcttcatcctcaaaggtgtcactcacctctcccacccatgtctcccccgacctttcctcctccaactactgtctctcccacctcagccgctacatagttcacctggggcagcacaacctccagaaggaggagggctgtgagcagacccggacagccactgagtccttcccccaccccggcttcaacaacagcctccccaacaaagaccaccgcaatgacatcatgctggtgaagatggcatcgccagtctccatcacctgggctgtgcgacccctcaccctctcctcacgctgtgtcactgctggcaccagctgcctcatttccggctggggcagcacgtccagcccccagtgtaggagcaccagaggggaacctggcagggggtggtgaggagggagtggtcaggattgtggaagggttcagggcatcagagatgcggttcacagtgacgatgtgggataagttgagaggatgtgtggaaaacgtcaggataggggggtggggacaaaagttggggccttggagtcagacggacgggatatgcaatcatacatccataacctcctggttgtaagaccttaggcaagcagcttcacctctctgaatcttgattttcttctctataaaatgagaatgattatacccacctgtcaggattggattagagataatgtatatcaagcaactgacataaatcatttattggatagcaggctgggcaccgtggctcacgcctgtaatcccagcactttgggaggccgaggtgggaagatcacctgaggtcaggactttgataccagcctggccaacgtggtgaaatcccatctctactaaaaatgtgaaaattagttgggcgtggttgtgtgcgcctgtaatcccagctactcgggaggttgaggcaggagaatcgcttaaacttgggagacggaggttgcagtgagccaagatcacgccactgcactccagcctgggcaacagagcaagactctgtctcgaaaaaaaaaaaaaaaaagctggatagcattgctgttgctattgttacaagaagagaggtgagttggctgcgtctaaggacagggattcccccaggggcgggatcacagcaagcactgcattaggggaggtggcagggggctcattcccacagcccctcacgctgtttccacagtacgcctgcctcacaccttgcgatgcgccaacatcaccatcattgagcaccagaagtgtgagaacgcctaccccggcaacatcacagacaccatggtgtgtgccagcgtgcaggaagggggcaaggactcctgccaggtcagtgtggtctccaaccacagccccatccccatccccagcttcaatgacatctttaccgacatccacaatttcatccccaacctcaacccgccgacccctgcaactcccaatccatctcttcccctgttcccgtttctgacctcagcacaaacttcagctccatccccgtttccacaccatttccagctccaaccatccccaaactcgtttttgagcctaaccccatcctttatcccacccataatcccagctttatcgctaaacctatcacctttcccagtgcctacccatcctgtctcggccccactcctaagcaccgtccccacctcctccctggctaacaccatgctcaacgctttctctgaccgacattctctctccccgtgcccagggtgactccgggggccctctggtctgtaaccagtctcttcaaggcattatctcctggggccaggatccgtgtgcgatcacccgaaagcctggtgtctacacgaaagtctgcaaatatgtggactggatccaggagacgatgaagaacaattagactggacccacccaccacagcccatcaccctccatttccacttggtgtttggttcctgttcactctgttaataagaaaccctaagccaagaccctctacgaacattctttgggcctcctggactacaggagatgctgtcacttaataatcaacctggggttcgaaatcagtgagacctggattcaaattctgccttgaaatattgtgactctgggaatgacaacacctggtttgttctctgttgtatccccagccccaaagacagctcctggccatatatcaaggtttcaataaatatttgctaaatgagtgaatctactgagtgcttactatgtgctagaccctgatccaatggcttttattttattttattttttgacagagtctcgctctgtcacccaggctggagtacagtggtgctatctctgctcactgcaacctccacctcctgggttcaagcaattctcctgcctcagcctcctgaatagctgggattacaggtgcctaccaccacatccggctaatttttgtattttttagtagagatggggcttcaccatgttggccaggctggtctcgaactcctgacctcagatgatctgccctccttggcctcccaaactcctgggattacagacgtgagccaccgcgcccgcccggctttcatttattaattaaaagaaattaaattaattaatctatttaggagacagtcttgctctgttgcccaggctggagtgcagtaacaatcacagctcacggcaatctcaatttcctggggtcaagtgattgtcctccctcagcctccagagtagctgggactacaggcacatgccacgaagcccagctaatttttgtatttttcgtagagacagaggtctcagtatgttgccccggctagtctcaaactcctgggctcaagcagtctgtcctcctcagcctccaaaagtggtgagattacaggcatgagtcgctgtgcctggcctccaagcactttcaaatgtatcaacttaatcctcacaaaaccctgtgaggtcggtactgttttcatacctattttatagttgaagaaacagacacagagaagcaaagtcacttgctcacagtcacgtggctaggagagcaaggatctgaagcaaggcgatctcttaattaccaagtgatgttcctggagtaaggctctgtttgtttcctttcctgtaaaatgctgcatgcaaaagtataacacagtaagtaaagaagtcagttagcctgcacatactaagacctaaccaaaggagcttattgtttttctccaacttccatgataggtaattagatagtggagacctctgctggccaatatggtagccactaaccgcagctggctcttccaattaaaattacataaagccagaaatgtaactcctctgtctcacttgttatatctccaaggctggatagccacatgtgactggtggtggctggattagctagtgcatataaaacatcactgcagaaagttcagctgagcagcactgagttagatggcctctgaagaggatgtcccacggagagaatccagaactcaggatctttttttttttttttctttgcgacagagtcttgctctgtcacccaggctggagtgcagtggcgtgatctcggctcactgcaacttctgcctcccaggttcaagcaattctcctgcctcagcctccctagtagctgggactacaggcctgtgccaacatccccagctaatttttgtgtctttttagtagagatggggtttcactatgttggccaggctggtctcgaactcctgacc

(19) United States
(12) Patent Application Publication
(10) Pub. No.: US 2005/0106586 A1
(43) Pub. Date: May 19, 2005

(54) Detection of neurodegenerative diseases

(75) Inventor: Eleftherios P. Diamandis, Toronto (CA)

Correspondence Address: MERCHANT & GOULD PC, P.O. BOX 2903, MINNEAPOLIS, MN, 55402-0903, US

(73) Assignee: MOUNT SINAI HOSPITAL, TORONTO (CA)

(21) Appl. No.: 10/868,490

(22) Filed: Jun. 14, 2004

Related U.S. Application Data
(60) Provisional application No. 60/478,486, filed on Jun. 13, 2003.

(51) Int. Cl. C12Q 1/68; G01N 33/53

(52) U.S. Cl. 435/6; 435/7.1

(57) ABSTRACT

The invention relates to compositions, kits, and methods for detecting, characterizing, preventing, and treating neurodegenerative diseases. In particular, the invention utilizes kallikrein 7 and kallikrein 10 and nucleic acids encoding same, to detect, characterize, prevent and treat neurodegenerative diseases.

### FIELD OF THE INVENTION

The invention relates to compositions, kits, and methods for detecting, characterizing, preventing, and treating neurodegenerative diseases.

### BACKGROUND OF THE INVENTION

The most common types of primary degenerative dementia are Alzheimer's disease (AD) and frontotemporal dementia (FTD). AD is typically characterised by a progressive decline in cognitive functions such as memory, abstract thinking, language comprehension and visuospatial functions ([Hardy and Selkoe, 2000; Selkoe, 2001; Sjogren, 1950; Sourander and Sjogren, 1970; Blennow and Wallin, 1992). Although cognitive changes also occur in FTD they do not prevail. Instead, changes in personality, affect, behaviour, self-control and monitoring are the typical features of FTD (Sjogren and Wallin, 2001). Both have a insidious onset and a continuous course leading to severe impairment and loss of independence (Pasquier, 1996). The etiology of AD and FTD are, in a few hereditary cases, well described and due to specific genetic alterations. However, in the vast majority of cases, the cause of AD and FTD is unknown. Although these disorders most probably are etiologically different, they may share some pathophysiological mechanisms, for example the involvement of certain structural proteins such as cytoskeleton proteins (Sjogren and Wallin, 2001; Pasquier, 1996; Sjogren et al. 2000). Other mechanisms have been proposed such as inflammatory changes, but the knowledge of the underlying mechanisms in AD and especially in FTD are unidentified (American Psychiatric Association 1987; Neary et al. 1998).

Recently, the amyloid hypothesis of Alzheimer's disease has been reviewed and updated (Hardy and Selkoe, 2002; Selkoe, 2002). It is thought that amyloid beta protein (Aβ-42), a 42 amino acid hydrophobic peptide generated by proteolytic digestion of amyloid precursor protein (APP) precipitates intracellularly, in several brain regions especially the hippocampus, interrupting the function of neurons. The role of various proteases, known as alpha, beta and gamma secretases in APP processing and in the pathogenesis of Alzheimer's disease is fairly-well documented. For reviews, see (Hardy and Selkoe, 2002; Selkoe, 2001; Selkoe, 2002).

Human tissue kallikreins are secreted serine proteases, encoded by genes that are tandemly localized on chromosome 19q13.4 (Diamandis et al., 2000a; Yousef and Diamandis, 2001). There are now 15 known members of the human tissue kallikrein family. It has already been demonstrated that some of these enzymes, including human kallikreins 5, 6, 7, 8, 9, 10, 11, 12 and 14 are expressed in the central nervous system (Yousef and Diamandis, 2001). Notably, human kallikrein 6 protein (hK6) has been identified at relatively very high levels in cerebrospinal fluid (up to 2 mg/L) (Diamandis et al., 2000b) and it has been previously associated with Alzheimer's disease (Diamandis et al., 2000; Zarghooni et al., 2002). KLK6 mRNA has been demonstrated in many brain regions and in spinal cord (Yousef et al., 1999) and hK6 enzyme has been immunohistochemically localized in choroid plexus epithelium, Purkinje cells and glial cells (Petraki et al., 2001). Mitsui et al. speculated that this kallikrein is related to aging and is a new risk factor for Alzheimer's disease and that its CSF concentration is reduced in some patients with AD (Mitsui et al., 2002). Previously, Little et al. have shown that this protease may have amyloidogenic potential (Little et al., 1997). Also, Scarsisbrick et al. postulated that myelencephalon-specific protease (MSP), which is identical to human kallikrein 6 (Scarisbrick et al., 2001) is highly expressed by inflammatory cells within the CNS and may promote demyelination (Scarisbrick et al., 2002). Another enzyme of this family, human kallikrein 8, is postulated to play a role in CNS function and in neural plasticity (Yoshida and Shiosaka, 1999). Previous studies have demonstrated expression of KLK7 in brain, spinal cord and cerebellum (Youssef et al., 2000a). Also, the KLK11 gene (previously known as trypsin-like serine protease or hippostasin) (Diamandis et al., 2000d) was cloned from hippocampal cDNA and is highly expressed in brain (Yoshida et al., 1998; Yousef et al., 2000b; Mitsui et al., 2000). More recently, Shimizu-Okabe et al. have reported a significant increase in KLK8 mRNA expression in hippocampal tissue from Alzheimer's disease patients, in comparison to controls (Shimizu-Okabe et al., 2001) while Ogawa et al. reported lower levels of KLK6 expression in brain of AD patients (Ogawa et al., 2000). Therapeutic strategies based on serine protease inhibitors, in preventing neuronal cell death, have been proposed (Rideout et al., 2001). A more detailed discussion on the role of serine proteases in the central nervous system has been published (Yoshida and Shiosaka, 1999) while another review summarizes the role of tissue kallikreins in the central nervous system (Yousef et al., 2003).

### SUMMARY OF THE INVENTION

The present invention seeks to overcome the drawbacks inherent in the prior art by providing sensitive and accurate methods for the detection of neurodegenerative diseases. It has been found that one or more kallikrein polypeptides wherein the kallikrein polypeptides comprise kallikrein 7 and kallikrein 10 and polynucleotides or nucleic acids encoding the polypeptides, have particular application in the detection of a neurodegenerative disease. Thus, the kallikrein markers constitute biomarkers for the diagnosis, monitoring, progression, treatment, and prognosis of neurodegenerative disease, and they may be used as biomarkers before surgery or after relapse.

In accordance with the methods of the invention, the presence of levels of kallikrein markers in a sample can be assessed, for example by detecting the presence in the sample of (a) polypeptides or polypeptide fragments corresponding to the markers; (b) metabolites which are produced directly or indirectly by polypeptides corresponding to the markers; (c) transcribed nucleic acids or fragments thereof having at least a portion with which the markers are substantially identical; and/or (c) transcribed nucleic acids or fragments thereof, wherein the nucleic acids hybridize with the markers.

In an embodiment, the invention provides a method for detecting one or more kallikrein polypeptides wherein the kallikrein polypeptides comprise or are selected from the group consisting of kallikrein 7 and kallikrein 10 associated with a neurodegenerative disease in a patient comprising:
(a) obtaining a sample from a patient;
(b) detecting or identifying in the sample one or more kallikrein polypeptides wherein the kallikrein polypeptides comprise or are selected from the group consisting of kallikrein 7 and kallikrein 10 associated with a neurodegenerative disease; and
(c) comparing the detected amounts with amounts detected for a standard.

The term “detect” or “detecting” includes assaying, assessing, imaging or otherwise establishing the presence or absence of the target kallikrein polypeptides or nucleic acids encoding the polypeptides, subunits thereof, or combinations of reagent bound targets, and the like, or assaying for, imaging, ascertaining, establishing, or otherwise determining one or more factual characteristics of a neurodegenerative disease. The term encompasses diagnostic, prognostic, and monitoring applications. The kallikrein markers can be detected individually, sequentially, or simultaneously.

According to a method involving one or more kallikrein polypeptides wherein the kallikrein polypeptides comprise kallikrein 7 and kallikrein 10 associated with a neurodegenerative disease, the levels in the sample of one or more kallikrein polypeptides wherein the kallikrein polypeptides comprise kallikrein 7 and kallikrein 10 associated with neurodegenerative disease, are compared with the normal levels of the kallikrein polypeptides, in samples of the same type obtained from controls (e.g. samples from individuals not afflicted with neurodegenerative disease). Significantly altered levels in the sample of the kallkrein polypeptides relative to the normal levels in a control is indicative of neurodegenerative disease.

In an embodiment, the invention provides a method for diagnosing and monitoring neurodegenerative disease in a subject comprising detecting in a sample from the subject one or more kallikrein polypeptides wherein the kallikrein polypeptides comprise kallikrein 7 and kallikrein 10 associated with neurodegenerative disease. The kallikrein markers can be detected using antibodies that bind to the kallikrein markers or parts thereof.

In a particular embodiment, a method is provided for diagnosing and monitoring Alzheimer's Disease in a subject comprising detecting in a sample from the subject one or more kallikrein polypeptides wherein the kallikrein polypeptides comprise kallikrein 7 and kallikrein 10 associated with Alzheimer's disease. In another particular embodiment, a method is provided for diagnosing and monitoring a frontotemporal dementia in a subject comprising detecting in a sample from the subject one or more kallikrein polypeptides wherein the kallikrein polypeptides comprise kallikrein 7 and kallikrein 10 associated with a frontotemporal dementia.

In an aspect the invention provides a method of assessing whether a patient is afflicted with or has a pre-disposition for a neurodegenerative disease, the method comprising comparing:
(a) levels of one or more of kallikrein polypeptides in a sample from the patient wherein the kallikrein polypeptides comprise kallikrein 7 and kallikrein 10 associated with the neurodegenerative disease; and
(b) normal levels of the kallikrein polypeptides, in samples of the same type obtained from control patients not afflicted with the neurodegenerative disease, wherein significantly altered levels of the kallikrein polypeptides, relative to the corresponding normal levels of the kallikrein polypeptides, is an indication that the patient is afflicted with neurodegenerative disease.

In an embodiment the invention provides a method of assessing whether a patient is afflicted with or has a pre-disposition for Alzheimer's Disease, the method comprising comparing:
(a) levels of one or both kallikrein polypeptides in a sample from the patient wherein the kallikrein polypeptides comprise kallikrein 7 and kallikrein 10 associated with Alzheimer's Disease; and
(b) normal levels of the kallikrein polypeptides, in samples of the same type obtained from control patients not afflicted with Alzheimer's Disease, wherein significantly lower levels of kallikrein 7 and/or higher levels of kallikrein 10, relative to the corresponding normal levels of the kallikrein polypeptides, is an indication that the patient is afflicted with Alzheimer's Disease.

In an embodiment the invention provides a method of assessing whether a patient is afflicted with or has a pre-disposition for frontotemporal dementia, the method comprising comparing:
(a) levels of one or both kallikrein polypeptides in a sample from the patient wherein the kallikrein polypeptides comprise kallikrein 7 and kallikrein 10 associated with frontotemporal dementia; and
(b) normal levels of the kallikrein polypeptides, in samples of the same type obtained from control patients not afflicted with frontotemporal dementia, wherein significantly lower levels of kallikrein 7 and/or lower levels of kallikrein 10, relative to the corresponding normal levels of the kallikrein polypeptides, is an indication that the patient is afflicted with frontotemporal dementia.

In another embodiment of a method of assessing whether a patient is afflicted with a neurodegenerative disease (e.g. screening, detection of a recurrence, reflex testing), the method comprises comparing:
(a) levels of one or more kallikrein polypeptides in a sample from the patient, wherein the kallikrein polypeptides comprise kallikrein 7 and kallikrein 10 associated with a neurodegenerative disease; and
(b) normal levels of the kallikrein polypeptides, in a control non-neurodegenerative disease sample.

A significant difference between the levels of the kallikrein polypeptides in the patient sample and the normal levels is an indication that the patient is afflicted with neurodegenerative disease.

The invention further relates to a method of assessing the efficacy of a therapy for inhibiting a neurodegenerative disease in a patient. This method comprises comparing:
(a) levels of one or more kallikrein polypeptides in a sample from the patient obtained from the patient prior to providing at least a portion of the therapy to the patient, wherein the kallikrein polypeptides comprise kallikrein 7 and kallikrein 10 associated with the neurodegenerative disease; and
(b) levels of the kallikrein polypeptides in a second sample obtained from the patient following therapy.

A significant difference between the levels of the kallikrein polypeptides in the second sample, relative to the first sample, is an indication that the therapy is efficacious for inhibiting neurodegenerative disease.

The “therapy” may be any therapy for treating neurodegenerative disease including but not limited to therapeutics, immunotherapy, gene therapy, and surgical removal of tissue. Therefore, the method can be used to evaluate a patient before, during, and after therapy.

In an aspect, the invention provides a method for monitoring the progression of a neurodegenerative disease in a patient, the method comprising:
(a) detecting in a patient sample at a first time point, one or more kallikrein polypeptides in a sample from the patient wherein the kallikrein polypeptides comprise kallikrein 7 and kallikrein 10 associated with a neurodegenerative disease; and
(b) repeating step (a) at a subsequent point in time; and
(c) comparing the levels detected in (a) and (b), and therefrom monitoring the progression of the neurodegenerative disease in the patient.

The invention also provides a method for assessing the potential efficacy of a test agent for inhibiting neurodegenerative disease in a patient, and a method of selecting an agent for inhibiting neurodegenerative disease in a patient.

The invention further provides a method of inhibiting neurodegenerative disease in a patient comprising:
(a) obtaining a sample comprising diseased cells from the patient;
(b) separately maintaining aliquots of the sample in the presence of a plurality of test agents;
(c) comparing levels of a one or more kallikrein polypeptides, in each of the aliquots, wherein the kallikrein polypeptides comprise kallikrein 7 and kallikrein 10 associated with neurodegenerative disease;
(d) administering to the patient at least one of the test agents which alters the levels of the kallikrein polypeptides in the aliquot containing that test agent, relative to other test agents.

The invention also contemplates a method of assessing the potential of a test compound to contribute to a neurodegenerative disease comprising:
(a) maintaining separate aliquots of neurodegenerative disease cells in the presence and absence of the test compound; and
(b) comparing levels of one or more kallikrein polypeptides in each of the aliquots, wherein the kallikrein polypeptides comprise kallikrein 7 and kallikrein 10.

A significant difference between the levels of the kallikrein polypeptides in the aliquot maintained in the presence of (or exposed to) the test compound relative to the aliquot maintained in the absence of the test compound, indicates that the test compound potentially contributes to the neurodegenerative disease.

In embodiments of the methods of the invention one or two kallikrein polypeptides are employed. In preferred embodiments, the kallikrein polypeptides comprise kallikrein 7; kallikrein 10; and kallikrein 7 and kallikrein 10.

Other methods of the invention employ one or more polynucleotides or nucleic acids capable of hybridizing to polynucleotides encoding kallikrein polypeptides wherein the kallikrein polypeptides comprise kallikrein 7 and kallikrein 10 associated with the neurodegenerative disease. Methods for detecting nucleic acids encoding kallikrein polypeptides can be used to monitor neurodegenerative disease by detecting the nucleic acids.

Thus, the present invention relates to a method for diagnosing and monitoring neurodegenerative disease in a sample from a subject comprising isolating nucleic acids, preferably mRNA, from the sample; and detecting nucleic acids encoding one or more kallikrein polypeptides in the sample, wherein the kallikrein polypeptides comprise kallikrein 7 and kallikrein 10. The presence of different levels of nucleic acids encoding the kallikrein polypeptides, in the sample compared to a standard or control is indicative of disease, disease stage, and/or prognosis, e.g. longer progression-free and overall survival.

In an embodiment, the invention provides methods for determining the presence or absence of a neurodegenerative disease in a subject comprising (a) contacting a sample obtained from the subject with oligonucleotides that hybridize to nucleic acids encoding one or more kallikrein polypeptides wherein the kallikrein polypeptides comprise kallikrein 7 and kallikrein 10; and (b) detecting in the sample levels of polynucleotides that hybridize to the nucleic acids relative to a predetermined cut-off value, and therefrom determining the presence or absence of the neurodegenerative disease in the subject. Within certain embodiments, mRNA is detected via polymerase chain reaction using, for example oligonucleotide primers that hybridize to nucleic acids encoding kallikrein polypeptides, or complements of such nucleic acids. Within other embodiments, the amount of mRNA is detected using a hybridization technique, employing oligonucleotide probes that hybridize to nucleic acids encoding kallikrein polypeptides, or complements of such nucleic acids.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of nucleic acids encoding kallikrein polypeptides in the sample. For mRNA the analyzing step may be accomplished using Northern Blot analysis to detect the presence of nucleic acids encoding kallikrein polypeptides. The analysis step may be further accomplished by quantitatively detecting the presence of nucleic acids encoding kallikrein polypeptides in the amplification product, and comparing the quantity of markers detected against a panel of expected values for the known presence or absence of the markers in normal and malignant tissue derived using similar primers.

The invention also provides a diagnostic composition comprising kallikrein polypeptides, or nucleic acids encoding the polypeptides, or agents that bind to the polypeptides or nucleic acids.

In an embodiment, the composition comprises probes that specifically hybridize to nucleic acids encoding kallikrein polypeptides, or fragments thereof. In another embodiment a composition is provided comprising specific primer pairs capable of amplifying nucleic acids encoding one or more kallikrein polypeptides, using polymerase chain reaction methodologies. In a still further embodiment, the composition comprises agents that bind to kallikrein polypeptides (e.g. antibodies) or fragments thereof. Probes, primers, and agents can be labeled with detectable substances.

In an aspect the invention provides an in vivo method comprising administering to a subject agents that have been constructed to target kallikrein polypeptides.

The invention therefore contemplates a method comprising administering to cells or tissues imaging agents that carry labels for imaging and that bind to kallikrein polypeptides, and then imaging the cells or tissues.

Still further the invention relates to therapeutic applications for neurodegenerative disease employing kallikrein polypeptides and nucleic acids encoding the polypeptides, and/or agents identified using methods of the invention.

The invention also includes kits for carrying out methods of the invention. In an embodiment, the kit is for assessing whether a patient is afflicted with a neurodegenerative disease and it comprises reagents for assessing one or more kallikrein polypeptides, wherein the kallikrein polypeptides comprise or are selected from the group consisting of kallikrein 7 and kallikrein 10.

In another aspect the invention relates to a kit for assessing the suitability of each of a plurality of test compounds for inhibiting a neurodegenerative disease in a patient. The kit comprises reagents for assessing kallikrein polypeptides, wherein the kallikrein polypeptides comprise or are selected from the group consisting of kallikrein 7 and kallikrein 10. The kit may also comprise a plurality of test agents or compounds.

The invention contemplates a kit for assessing the presence of neurodegenerative disease cells, wherein the kit comprises antibodies specific for one or more selected markers, wherein the markers comprise or are selected from the group consisting of kallikrein 7 and kallikrein 10.

Additionally the invention provides a kit for assessing the potential of a test compound to contribute to a neurodegenerative disease. The kit comprises neurodegenerative disease cells and reagents for assessing one or more kallikrein markers, wherein the markers comprise or are selected from the group consisting of kallikrein 7 and kallikrein 10.

In an aspect the invention provides a method of treating a patient afflicted with neurodegenerative disease comprising providing to cells of a patient antisense oligonucleotides complementary to nucleic acids encoding one or more kallikrein polypeptides, which are overexpressed in neurodegenerative disease. In an alternative method, expression of genes corresponding to one or more kallikrein polypeptides which are underexpressed in neurodegenerative disease are increased.

The invention relates to a method of inhibiting neurodegenerative disease in a patient at risk for developing neurodegenerative disease comprising inhibiting or increasing expression (or overexpression) of genes encoding one or more kallikrein polypeptides, wherein the kallikrein polypeptides comprise one or more of or are selected from the group consisting of kallikrein 7 and kallikrein 10 that are either overexpressed or underexpressed, in neurodegenerative disease.

The invention also contemplates the methods, compositions, and kits described herein using additional markers for neurogenerative diseases. Additional markers include markers to kallikrein 6, kallikrein 11, KLK6, and KLK11 (See Table 5 and SEQ ID Nos. 7 through 14). Preferably the other markers are markers to kallikrein 6 or KLK6. The methods described herein may be modified by including reagents to detect the additional markers, or nucleic acids for the markers.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1: Distribution of CSF hK6 concentration in control subjects and patients with frontotemporal dementia and Alzheimer's disease. N=number of subjects per group. The horizontal lines indicate median levels. For statistical comparisons, see Table 2.

FIG. 2: Distribution of CSF hK7 concentration in control subjects and patients with frontotemporal dementia and Alzheimer's disease. N=number of subjects per group. The horizontal lines indicate median levels. For statistical comparisons, see Table 2.

FIG. 3: Distribution of CSF hK10 concentration in control subjects and patients with frontotemporal dementia and Alzheimer's disease. N=number of subjects per group. The horizontal lines indicate median levels. For statistical comparisons, see Table 2.

FIG. 4: Correlation of CSF concentration of hK6 and hK7 in the whole group of subjects (N=51). The Pearson correlation coefficient was 0.65.

### DETAILED DESCRIPTION OF THE INVENTION

The invention relates to newly discovered correlations between expression of certain markers and neurodegenerative disease. The combinations of markers described herein provide sensitive methods for detecting neurodegenerative disease. The levels of expression of a combination of markers described herein correlates with the presence of a neurodegenerative disease in a patient. Methods are provided for detecting the presence of a neurodegenerative disease in a sample, the absence of a neurodegenerative disease in a sample, assessing the histology of tissues associated with a neurodegenerative disease, and other characteristics of a neurodegenerative disease that are relevant to prevention, diagnosis, characterization, and therapy of a neurodegenerative disease in a patient. Methods are also provided for assessing the efficacy of one or more test agents for inhibiting kallikreins that affect a neurodegenerative disease, assessing the efficacy of a therapy for neurodegenerative disease, monitoring the progression of a neurodegenerative disease, selecting an agent or therapy for inhibiting a neurodegenerative disease, treating a patient afflicted with neurodegenerative disease, inhibiting a neurodegenerative disease in a patient, and assessing the potential of a test compound to contribute to a neurodegenerative disease.

Glossary

The terms “sample”, “biological sample”, and the like, mean a material known or suspected of expressing or containing kallikrien polypeptides associated with a neurodegenerative disease, or nucleic acids encoding the polypeptides. The test sample can be used directly as obtained from the source or following a pretreatment to modify the character of the sample. The sample can be derived from any biological source, such as tissues, extracts, or cell cultures, including cells, cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Nucleic acids and polypeptides may be isolated from the samples and utilized in the methods of the invention. In a preferred embodiment, the sample is cerebral spinal fluid or serum.

The term “subject” or “patient” refers to a warm-blooded animal such as a mammal, which is suspected of having a neurodegenerative disease. Preferably, “subject” refers to a human.

“Neurodegenerative Disease” include Alzheimer's Disease and other tauopathies such as frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-park-insonism-amytrophy complex, Pick's disease, or Pick's disease-like dementia.

“Kallikrein polypeptides” or “kallikrein markers” comprises one or both of kallikrein 7 and kallikrein 10, and optionally one or both of kallikrein 6 and kallikrein 11. The term includes the native-sequence polypeptides, isoforms, precursors and chimeric polypeptides. The amino acid sequences for native kallikrein polypeptides employed in the present invention include the sequences found in GenBank for each polypeptide as shown in Table 5, and in SEQ ID NO: 1 (kallikrein 7), NO. 4 (kallikrein 10), NO. 7 (kallikrein 6), NO. 12 (kallikrein 11), or a portion thereof. Other useful polypeptides are substantially identical to these sequences (e.g. at least about 45%, preferably 50%, 55%, 60%, 65%, 70%, 75%, 80%, 85%, 90%, 95%, 97%, 98%, or 99% sequence identity), and preferably retain the immunogenic activity of the corresponding native-sequence kallikrein polypeptide.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g., alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95% amino acid sequence identity to the sequences identified in the GenBank Accession Nos. in Table 5 and shown in SEQ ID NOS. 1, 4, 7, 11, and 12. Such variants include, for instance, polypeptides wherein one or more amino acid residues are added to, or deleted from, the N- or C-terminus of the full-length or mature sequences of SEQ ID NOS: 1, 4, 7, 11, and 12, including variants from other species, but excludes a native-sequence polypeptide.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an animo acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a kallikrein polypeptide homolog, for example, the murine kallikrein polypeptide.

Percent identity of two amino acid sequences, or of two nucleic acid sequences identified herein is defined as the percentage of amino acid residues or nucleotides in a candidate sequence that are identical with the amino acid residues in a kallikrein polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

Kallikrein polypeptides include chimeric or fusion proteins. A “chimeric protein” or “fusion protein” comprises all or part (preferably biologically active) of a kallikrein polypeptide operably linked to a heterologous polypeptide (i.e., a polypeptide other than the same kallikrein polypeptide). Within the fusion protein, the term “operably linked” is intended to indicate that the kallikrein polypeptide and the heterologous polypeptide are fused in-frame to each other. The heterologous polypeptide can be fused to the N-terminus or C-terminus of the kallikrein polypeptide. A useful fusion protein is a GST fusion protein in which a kallikrein polypeptide is fused to the C-terminus of GST sequences. Another example of a fusion protein is an immunoglobulin fusion protein in which all or part of a kallikrein polypeptide is fused to sequences derived from a member of the immunoglobulin protein family. Chimeric and fusion proteins can be produced by standard recombinant DNA techniques.

Kallikrein polypeptides may be isolated from a variety of sources, such as from human tissue types or from another source, or prepared by recombinant or synthetic methods, or by any combination of these and similar techniques.

“Kallikrein polynucleotides” refers to one or both of kallilkrein 7 polynucleotide (KLK7), kallikrein 10 polynucleotide (KLK10), and optionally one or both of kallikrein 6 polynucleotide (KLK6) and kallikrein 11 polynucleotide (KLK11). The term includes nucleic acids that encode a native-sequence polypeptide, a polypeptide variant including a portion of a kallikrein polypeptide, an isoform, precursor, and chimeric polypeptide.

The nucleic acid sequences encoding native kallikrein polypeptides employed in the present invention include the nucleic acid sequences of the GenBank Accession Nos. identified in Table 5, and in SEQ ID NOs: 2 and 3 (KLK7), NOs. 5 and 6 (KLK10), NOs. 8 through 10 (KLK6), and NOs 13 and 14 (KLK11), or fragments thereof.

Polynucleotides encoding kallikrien polypeptides include nucleic acid sequences complementary to these nucleic acids, and nucleic acids that are substantially identical to these sequences (e.g. at least about 45%, preferably 50%, 55%, 60%, 65%, 70%, 75%, 80%, 85%,90%, 95%, 97%, 98%, or 99% sequence identity).

Kallikrein polynucleotides also include sequences which differ from a nucleic acid sequence of the GenBank Accession Nos. identified in Table 5 and SEQ ID NOS: 2, 3, 5, 6, 8-10, 13, and 14, due to degeneracy in the genetic code. As one example, DNA sequence polymorphisms within the nucleotide sequence of a kallikrein polypeptide may result in silent mutations that do not affect the amino acid sequence. Variations in one or more nucleotides may exist among individuals within a population due to natural allelic variation. DNA sequence polymorphisms may also occur which lead to changes in the amino acid sequence of a kallikrein polypeptide.

Kallikrein polynucleotides also include nucleic acids that hybridize under stringent conditions, preferably high stringency conditions to a nucleic acid sequence of the GenBank Accession Nos. identified in Table 5 and SEQ ID NOS: 2, 3, 5, 6, 8-10, 13, and 14. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

Kallikrein polynucleotides also include truncated nucleic acids or nucleic acid fragments and variant forms of the nucleic acids that arise by alternative splicing of an mRNA corresponding to a DNA.

Kallikrien polynucleotides are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method.

One or more kallikrein polypeptides or kallikrein polynucleotides are detected in the present invention. In one aspect, the kallikrein polypeptides comprise kallikrein 7; kallikrein 10; kallikrein 7 and kallikrein 10; kallikrein 6, kallikrein 7 and kallikrein 10. In another aspect, the kallikrein polynucleotides comprise KLK7; KLK10; KLK7 and KLK10; or KLK6, KLK7 and KLK10.

General Methods

A variety of methods can be employed for the diagnostic and prognostic evaluation of a 5 neurodegenerative disease involving kallikrein polypeptides, and polynucleotides encoding the polypeptides, and the identification of subjects with a predisposition to such disorders. Such methods may, for example, utilize polynucleotides encoding kallikrein polypeptides, and fragments thereof, and binding agents (e.g. antibodies) against kallikrein polypeptides, including peptide fragments. In particular, the polynucleotides and antibodies may be used, for example, for (1) the detection of either over- or under-expression of kallikrein polynucleotides, relative to a non-disorder state; and (2) the detection of either an over- or an under-abundance of kallikrein polypeptides, relative to a non-disorder state or the presence of modified (e.g., less than full length) kallikrein polypeptides that correlate with a disorder state, or a progression toward a disorder state.

The invention also contemplates a method for detecting a neurodegenerative disease comprising producing a profile of levels of kallikrein polypeptides in cells from a patient, wherein the markers are kallikrein 7 and kallikrein 10, and comparing the profile with a reference to identify a protein profile for the test cells indicative of disease.

The methods described herein may be used to evaluate the probability of the presence of a neurodegenerative disease for example, in a group of cells freshly removed from a host. Such methods can be used to detect disease, and help in the diagnosis and prognosis of disease. The methods can be used to confirm the absence or removal of all diseased tissue following surgery, and/or therapy. They can further be used to monitor therapy.

The methods described herein can be adapted for diagnosing and monitoring a degenerative disease by detecting kallikrein polypeptides, or polynucleotides encoding the polypeptides in biological samples from a subject. These applications require that the amount of polypeptides or polynucleotides quantitated in a sample from a subject being tested be compared to a predetermined standard. The standard may correspond to levels quantitated for another sample or an earlier sample from the subject, or levels quantitated for a control sample. Levels for control samples from healthy subjects or patients with disease may be established by prospective and/or retrospective statistical studies. Healthy or normal subjects who have no clinically evident disease or abnormalities may be selected for statistical studies. Diagnosis may be made by a finding of statistically different levels of kallikrein polypeptides, or nucleic acids encoding same, compared to a control sample or previous levels quantitated for the same subject.

“Statistically different levles” or “significant differences” in levels of markers in a patient sample compared to a control or standard (e.g. normal levels or levels in other samples from a patient) may represent levels that are higher or lower than the standard error of the detection assay, preferably the levels are at least about 1.5, 2, 3, 4, 5, or 6 times higher or lower, respectively, than the control or standard.

Protein Methods

Binding agents specific for kallikrein polypeptides may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of a neurodegenerative disease in a subject may be determined by (a) contacting a sample from the subject with binding agents for kallikrein polypeptides; (b) detecting in the sample levels of polypeptides that bind to the binding agents; and (c) comparing the levels of polypeptides with a predetermined standard or cut-off value.

“Binding agent” refers to a substance such as a polypeptide or antibody that specifically binds to a kallikrein polypeptide. A substance “specifically binds” to a polypeptide if it reacts at a detectable level with the kallikrein polypeptide, and does not react detectably with peptides containing unrelated sequences or sequences of different polypeptides. Binding properties may be assessed using an ELISA, which may be readily performed by those skilled in the art (see for example, Newton et al, Develop. Dynamics 197: 1-13, 1993).

A binding agent may be a ribosome, with or without a peptide component, an RNA molecule, or a polypeptide. A binding agent may be a polypeptide that comprises a kallikrein polypeptide sequence, a peptide variant thereof, or a non-peptide mimetic of such a sequence. By way of example a kallikrein polypeptide sequence may be a peptide portion of a kallikrein polypeptide that is capable of modulating a function mediated by the kallikrein polypeptide.

In certain other preferred embodiments, the binding agent is an antibody.

In an aspect the present invention provides a diagnostic method for monitoring or diagnosing a neurodegenerative disease in a subject by quantitating kallikrein polypeptides, in a biological sample from the subject comprising reacting the sample with antibodies specific for kallikrein polypeptides, which are directly or indirectly labelled with detectable substances, and detecting the detectable substances.

In an aspect of the invention, a method for detecting a neurodegenerative disease is provided comprising:
(a) obtaining a sample suspected of containing kallikrein polypeptides associated with a neurodegenerative disease, wherein the kallikrein polypeptides comprise or are selected from the group consisting of kallikrein 7 and kallikrein 10;
(b) contacting said sample with antibodies that specifically bind kallikrein polypeptides, under conditions effective to bind the antibodies and form complexes;
(c) measuring the amount of kallikrein polypeptides, present in the sample by quantitating the amount of the complexes; and
(d) comparing the amount of kallikrein polypeptides, present in the samples with the amount of polypeptides in a control, wherein a change or significant difference in the amount of polypeptides in the sample compared with the amount in the control is indicative of a neurodegenerative disease.

In an embodiment, the invention contemplates a method for monitoring the progression of a neurodegenerative disease in a subject, comprising:
(a) contacting antibodies which bind to kallikrein polypeptides, with a sample from the individual so as to form binary complexes comprising each of the antibodies and polypeptides in the sample, wherein the kallikrein polypeptides comprise or are selected from the group consisting of kallikrein 7 and kallikrein 10;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of the stage and/or progression of the neurodegenerative disease in said individual.

The amount of complexes may also be compared to a value representative of the amount of the complexes from an individual not at risk of, or afflicted with, a neurodegenerative disease at different stages.

Thus, antibodies specifically reactive with a kallikrein polypeptide, or derivatives, such as enzyme conjugates or labeled derivatives, may be used to detect a kallikrein polypeptide, in various samples (e.g. biological materials). They may be used as diagnostic or prognostic reagents and they may be used to detect abnormalities in the levels of expression of kallikrein polypeptides, or abnormalities in the structure, and/or temporal, tissue, cellular, or subcellular location of kallikrein polypeptides. Antibodies may also be used to screen potentially therapeutic compounds in vitro to determine their effects on neurodegenerative diseases involving kallikrein polypeptides, and other conditions. In vitro immunoassays may also be used to assess or monitor the efficacy of particular therapies.

Antibodies may be used in any known immunoassays that rely on the binding interaction between antigenic determinants of kallikrein polypeptides, and the antibodies. Examples of such assays are radioimmunoassays, enzyme immunoassays (e.g. ELISA), immunofluorescence, immunoprecipitation, latex agglutination, hemagglutination, and histochemical tests. These terms are well understood by those skilled in the art. A person skilled in the art will know, or can readily discern, other immunoassay formats without undue experimentation.

In particular, the antibodies may be used in immunohistochemical analyses, for example, at the cellular and sub-subcellular level, to detect kallikrein polypeptides, to localize them to particular cells and tissues, and to specific subcellular locations, and to quantitate the level of expression.

Antibodies for use in the present invention include monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant kallikrein polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. The antibodies specific for kallikrein polypeptides used in the methods of the invention may also be obtained from scientific or commercial sources.

In an embodiment of the invention, antibodies are reactive against kallikrein polypeptides if they bind with a Kₐ of greater than or equal to 10⁻¹ M.

Antibodies that bind to kallikrein polypeptides may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against a kallikrein polypeptide. The second antibody may be labeled with a detectable substance to detect the primary antigen-antibody reaction. By way of example, if the antibody having specificity against a kallikrein polypeptide is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect kallikrein polypeptides. Generally, antibodies may be labeled with detectable substances and kallikrein polypeptides, may be localised in tissues and cells based upon the presence of the detectable substance.

In the context of the methods of the invention, the sample, binding agents (e.g. antibodies) for kallikrein polypeptides may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for a kallikrein polypeptide may be immobilized using sheep anti-mouse IgG Fc fragment specific antibody coated on the carrier or support.

Where radioactive labels are used as a detectable substance, kallikrein polypeptides may be localized by radioautography. The results of radioautography may be quantitated by determining the density of particles in the radioautographs by various optical methods, or by counting the grains.

Time-resolved fluorometry may be used to detect a signal. For example, the method described in Christopoulos T K and Diamandis E P Anal Chem 1992:64:342-346 may be used with a conventional time-resolved fluorometer.

Therefore, in accordance with an embodiment of the invention, a method is provided wherein antibodies specific for each kallikrein polypeptide, are labelled with enzymes, substrates for the enzymes are added wherein the substrates are selected so that the substrates, or a reaction product of the enzymes and substrates, form fluorescent complexes with lanthanide metals. Lanthanide metals are added and the kallikrein polypeptides are quantitated in the sample by measuring fluorescence of the fluorescent complexes. Antibodies specific for the kallikrein polypeptides may be directly or indirectly labelled with enzymes. Enzymes are selected based on the ability of a substrate of the enzyme, or a reaction product of the enzyme and substrate, to complex with lanthanide metals such as europium and terbium. Examples of suitable enzymes include alkaline phosphatase and β-galactosidase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

Antibodies specific for kallikrein polypeptides may also be indirectly labelled with enzymes. For example, an antibody may be conjugated to one partner of a ligand binding pair, and the enzyme may be coupled to the other partner of the ligand binding pair. Representative examples include avidin-biotin, and riboflavin-riboflavin binding protein. In another embodiment, antibodies specific for the anti-kallikrein antibodies are labeled with an enzyme.

In accordance with an embodiment, the present invention provides means for determining kallikrein polypeptides in a sample, in particular a CSF or serum sample, by measuring kallikrein polypeptides by immunoassay. It will be evident to a skilled artisan that a variety of immunoassay methods can be used to measure kallikrein polypeptides in serum. In general, an immunoassay method may be competitive or noncompetitive. Competitive methods typically employ immobilized or immobilizable antibodies to the kallikrein polypeptides and a labeled form of each of the kallikrein polypeptides. Kallikrein polypeptides and labeled kallikrein polypeptides compete for binding to anti-kallikrein antibodies. After separation of the resulting labeled kallikrein polypeptides that have become bound to anti-kallikrein polypeptides (bound fraction) from that which has remained unbound (unbound fraction), the amount of the label in either bound or unbound fraction is measured and may be correlated with the amount of kallikrein polypeptides, in the test sample in any conventional manner, e.g., by comparison to a standard curve.

In an aspect, a non-competitive method is used for the determination of kallikrein polypeptides with the most common method being the “sandwich” method. In this assay, two types of antibodies specific for kallikrein polypeptides are employed. One type of antibody is directly or indirectly labeled (sometimes referred to as the “detection antibody”) and the other is immobilized or immobilizable (sometimes referred to as the “capture antibody”). The capture and detection antibodies can be contacted simultaneously or sequentially with a test sample. Sequential methods can be accomplished by incubating capture antibodies with the sample, and adding the detection antibodies at a predetermined time thereafter (sometimes referred to as the “forward” method); or the detection antibodies can be incubated with the sample first and then the capture antibodies added (sometimes referred to as the “reverse” method). After the necessary incubation(s) have occurred, to complete the assay, the capture antibodies are separated from the liquid test mixture, and labels are measured in at least a portion of the separated capture antibody phase or the remainder of the liquid test mixture. Generally the labels are measured in the capture antibody phase since it comprises kallikrein polypeptides bound by (“sandwiched” between) the capture and detection antibodies. In an embodiment, the label may be measured without separating the capture antibodies and liquid test mixture.

In a typical two-site immunometric assay for kallikrein polypeptides, one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The labels used with the detection antibodies can be selected from any of those known conventionally in the art. The labels may be an enzyme or a chemiluminescent moiety, but it can also be a radioactive isotope, a fluorophor, a detectable ligand (e.g., detectable by a secondary binding by a labeled binding partner for the ligand), and the like. Preferably antibodies are labelled with enzymes which are detected by adding substrates that are selected so that a reaction product of the enzymes and substrates forms fluorescent complexes. Capture antibodies may be selected so that they provide a means for being separated from the remainder of the test mixture. Accordingly, the capture antibodies can be introduced to the assay in an already immobilized or insoluble form, or can be in an immobilizable form, that is, a form which enables immobilization to be accomplished subsequent to introduction of the capture antibodies to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, a capture antibody may be immobilized using a species specific antibody for the capture antibody that is bound to the solid phase.

A particular sandwich immunoassay method of the invention employs two types of antibodies, first antibodies are reactive against kallikrein polypeptides and second antibodies having specificity against antibodies reactive against kallikrein polypeptides labelled with enzymatic labels, and fluorogenic substrates for the enzymes. An enzyme may be alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb3+ fluorescence in a time-resolved mode. Fluorescence intensity is measured using a time-resolved fluorometer as described herein.

The above-described immunoassay methods and formats are intended to be exemplary and are not limiting.

Nucleic Acid Methods/Assays

As noted herein a neurodegenerative disease may be detected based on the level of polynucleotides encoding kallikrein polypeptides in a sample. Techniques for detecting nucleic acid molecules such as polymerase chain reaction (PCR) and hybridization assays are well known in the art.

Nucleotide probes for use in the detection of nucleic acid sequences in samples may be constructed using conventional methods known in the art. Suitable probes may be based on nucleic acid sequences encoding at least 5 sequential amino acids from regions of nucleic acids encoding kallikrein polypeptides, preferably they comprise 15 to 40 nucleotides. A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect nucleic acids encoding kallikrein polypeptides preferably in human cells. The nucleotide probes may also be useful in the diagnosis of a neurodegenerative disease involving nucleic acids encoding kallikrein polypeptides, in monitoring the progression of such disorder; or monitoring a therapeutic treatment.

Probes may be used in hybridization techniques to detect polynucleotides encoding kallikrein polypeptides. The technique generally involves contacting and incubating nucleic acids (e.g. recombinant DNA molecules, cloned genes) obtained from a sample from a patient or other cellular source with probes under conditions favorable for the specific annealing of the probes to complementary sequences in the nucleic acids. After incubation, the non-annealed nucleic acids are removed, and the presence of nucleic acids that have hybridized to the probe if any are detected.

The detection of nucleic acids encoding kallikrein polypeptides, may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, oligonucleotide primers may be employed in a PCR based assay to amplify a portion of nucleic acids encoding kallikrein polypeptides, derived from a sample, wherein the oligonucleotide primers are specific for (i.e. hybridize to) nucleic acids encoding each of the kallikrein polypeptides. The amplified cDNA is then separated and detected using techniques well known in the art, such as gel electrophoresis.

In order to maximize hybridization under assay conditions, primers and probes employed in the methods of the invention generally have at least about 60%, preferably at least about 75% and more preferably at least about 90% identity to a portion of nucleic acids encoding kallikrein polypeptides; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of expression of polynucleotides encoding kallikrein polypeptides. For example, RNA may be isolated from a cell type or tissue known to express these polynucleotides and tested utilizing the hybridization (e.g. standard Northern analyses) or PCR techniques referred to herein.

The primers and probes may be used in the above-described methods in situ i.e directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample tissue using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to sets of primers specifically designed against a kallikrein polypeptide sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with a suspected neurodegenerative disease and an individual who is not afflicted with a neurodegenerative disease. The reaction may be performed on several dilutions of cDNA spanning at least two orders of magnitude. A statistically significant difference in expression in several dilutions of the subject sample as compared to the same dilutions of the non-disease sample may be considered positive for the presence of a neurodegenerative disease.

Oligonucleotides or longer fragments derived from nucleic acids encoding each kallikrein polypeptide may be used as targets in a microarray. The microarray can be used to simultaneously monitor the expression levels of large numbers of genes. The information from the microarray may be used to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

Thus the invention also includes an array comprising kallikrein marker(s). The array can be used to assay expression of kallikrein polynucleotides in the array. The invention allows the quantitation of expression of kallikrein markers.

In an embodiment, the array can be used to monitor the time course of expression of kallikrein nucleic acids in the array. This can occur in various biological contexts such as disease progression.

The array is also useful for ascertaining differential expression patterns of kallikrein polynucleotides in normal and abnormal cells. This provides a battery of nucleic acids that could serve as molecular targets for diagnosis or therapeutic intervention.

Computer Systems

Computer readable media comprising kallikrein markers is also provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on kallikrein markers.

A variety of data processor programs and formats can be used to store information on kallikrein markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has a neurodegenerative disease or a pre-disposition to a neurodegenerative disease, comprising determining the presence or absence of kallikrein markers, and based on the presence or absence of the kallikrein markers, determining whether the patient has a neurodegenerative disease or a pre-disposition to a neurodegenerative disease, and optionally recommending treatment for the neurodegenerative disease or pre-disease condition.

The invention also provides in an electronic system and/or in a network, a method for determining whether a subject has a neurodegenerative disease or a pre-disposition to a neurodegenerative disease associated with kallikrein markers, comprising determining the presence or absence of kallikrein markers, and based on the presence or absence of the kallikrein markers, determining whether the subject has a neurodegenerative disease or a pre-disposition to a neurodegenerative disease, and optionally recommending treatment for the a neurodegenerative disease or pre-disease condition.

The invention further provides in a network, a method for determining whether a subject has a neurodegenerative disease or a pre-disposition to a neurodegenerative disease associated with kallikrein markers, comprising: (a) receiving phenotypic information on the subject and information on kallikrein markers associated with samples from the subject; (b) acquiring information from the network corresponding to the kallikrein markers; and (c) based on the phenotypic information and information on the kallikrein markers determining whether the subject has a neurodegenerative disease or a pre-disposition to a neurodegenerative disease; and (d) optionally recommending treatment for the neurodegenerative disease or pre-disease condition.

The invention still further provides a system for identifying selected records that identify a neurodegenerative disease cell or tissue. A system of the invention generally comprises a digital computer; a database server coupled to the computer; a database coupled to the database server having data stored therein, the data comprising records of data comprising kallikrein markers, or nucleic acids encoding same, and a code mechanism for applying queries based upon a desired selection criteria to the data file in the database to produce reports of records which match the desired selection criteria.

In an aspect of the invention a method is provided for detecting a neurodegenerative disease tissue or cell using a computer having a processor, memory, display, and input/output devices, the method comprising the steps of:
(a) creating records of kallikrein markers isolated from a sample suspected of containing a neurodegenerative disease cell or tissue;
(b) providing a database comprising records of data comprising kallikrein markers, wherein the markers are kallikrein 7, and kallikrein 10, optionally one or both of kallikrein 6 and kallikrein 11, and/or comprising nucleic acids encoding same; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from a cell or tissue that is a neurodegenerative disease cell or tissue.

The invention contemplates a business method for determining whether a subject has a neurodegenerative disease or a pre-disposition to a neurodegenerative disease associated with kallikrein markers comprising: (a) receiving phenotypic information on the subject and information on kallikrein markers associated with samples from the subject; (b) acquiring information from a network corresponding to the kallikrein markers; and (c) based on the phenotypic information, information on the kallikrein markers, and acquired information, determining whether the subject has a neurodegenerative disease or a pre-disposition to a neurodegenerative disease; and (d) optionally recommending treatment for the a neurodegenerative disease or pre-condition.

Imaging Methods

Antibodies specific for kallikrein polypeptides may also be used in imaging methodologies in the management of a neurodegenerative disease. The invention provides a method for imaging tissues of subject with a neurodegenerative disease associated with kallikrein polypeptides.

In an embodiment the method comprises administering to a tissue of a subject with a neurodegenerative disease imaging agents that carry imaging labels and are capable of targeting or binding to kallikrein polypeptides. In the method each imaging agent is labeled so that it can be distinguished during the imaging. The imaging agents are allowed to incubate and bind to the kallikrein polypeptides. The presence of label is localized to the kallikreins associated with a neurodegenerative disease, and the localized label is detected using imaging devices known to those skilled in the art.

The imaging agents may be antibodies or chemical entities that recognize kallikrein polypeptides. In an aspect of the invention an imaging agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for kallikrein polypeptides used in the methods of the invention may be obtained from scientific or commercial sources, or isolated native or recombinant kallikrein polypeptides may be utilized to prepare antibodies etc as described herein.

An imaging agent may be a peptide that mimics the epitope for an antibody specific for a kallikrein polypeptide and binds to a kallikrein polypeptide. The peptide may be produced on a commercial synthesizer using conventional solid phase chemistry. By way of example, a peptide may be prepared that includes tyrosine, lysine, or phenylalanine to which N₂S₂ chelate is complexed (See U.S. Pat. No. 4,897,255). The anti-kallikrein peptide conjugate is then combined with a radiolabel (e.g. sodium ^{99m}Tc pertechnetate or sodium ¹⁸⁸Re perrhenate) and it may be used to locate a tumor producing kallikrein polypeptides.

Imaging agents carry labels to image the kallikrein polypeptides. Agents may be labelled for use in radionuclide imaging. In particular, agents may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe. ⁵⁵Fe, ⁵⁹Fe, ¹⁷⁷Lu, 15O, ^{190m-191}Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, 90Y, ⁶²Zn and ⁶⁵Zn. Preferably the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F, and is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for murine monoclonal antibodies are described by Goding, J. W. (supra, pp 124-126) and the references cited therein. Other procedures for iodinating biological agents, such as antibodies, binding portions thereof, probes, or ligands, are described in the scientific literature (see Hunter and Greenwood, Nature 144:945 (1962), David et al., Biochemistry 13:1014-1021 (1974), and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat. No. 4,311,688. Procedures suitable for ¹¹¹ In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

Screening Methods

The invention also contemplates methods for evaluating test agents or compounds for their ability to inhibit a neurodegenerative disease or potentially contribute to a neurodegenerative disease. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)2, and Fab expression library fragments, and epitope-binding fragments thereof)], and small organic or inorganic molecules. The agents or compounds may be endogenous physiological compounds or natural or synthetic compounds.

The invention provides a method for assessing the potential efficacy of a test agent for inhibiting a neurodegenerative disease in a patient, the method comprising comparing:
(a) levels of kallikrein markers in a first sample obtained from a patient and exposed to the test agent, wherein the markers comprise or are selected from the group consisting of kallikrein 7 and kallikrein 10, and
(b) levels of the kallikrein markers in a second sample obtained from the patient, wherein the sample is not exposed to the test agent, wherein a significant difference in the levels of expression of kallikrein markers in the first sample, relative to the second sample, is an indication that the test agent is potentially efficacious for inhibiting a neurodegenerative disease in the patient.

The first and second samples may be portions of a single sample obtained from a patient or portions of pooled samples obtained from a patient.

In an aspect, the invention provides a method of selecting an agent for inhibiting a neurodegenerative disease in a patient comprising:
(a) obtaining a sample from the patient;
(b) separately maintaining aliquots of the sample in the presence of a plurality of test agents;
(c) comparing kallikrein markers or nucleic acids encoding same, in each of the aliquots, wherein the markers comprise or are selected from the group consisting of kallikrein 7 and kallikrein 10; and
(d) selecting one of the test agents which alters the levels of the kallikrein markers or nucleic acids encoding same in the aliquot containing that test agent, relative to other test agents.

Still another aspect of the present invention provides a method of conducting a drug discovery business comprising:
(a) providing one or more methods or assay systems for identifying agents that inhibit a neurodegenerative disease in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test substance to contribute to a neurodegenerative disease comprising:
(a) maintaining separate aliquots of cells or tissues from a patient with a neurodegenerative disease in the presence and absence of the test compound; and
(b) comparing kallikrein markers or nucleic acids encoding same in each of the aliquots, wherein the markers comprise or are selected from the group consisting of kallikrein 7 and kallikrein 10.

A significant difference between the levels of the markers in the aliquot maintained in the presence of (or exposed to) the test compound relative to the aliquot maintained in the absence of the test compound, indicates that the test compound possesses the potential to contribute to a neurodegenerative disease.

Kits

The methods described herein may be performed by utilizing pre-packaged diagnostic kits comprising at least kallikrein polynucleotides or binding agents (e.g. antibodies) described herein, which may be conveniently used, e.g., in clinical settings, to screen and diagnose patients, and to screen and identify those individuals afflicted with or exhibiting a predisposition to a neurodegenerative disease.

Thus, the invention also contemplates kits for carrying out the methods of the invention. Such kits typically comprise two or more components required for performing a diagnostic assay. Components include but are not limited to compounds, reagents, containers, and/or equipment.

In an embodiment, a container with a kit comprises binding agents as described herein. By way of example, the kit may contain antibodies specific for kallikrein polypeptides, antibodies against the antibodies labelled with enzymes, and substrates for the enzymes. The kit may also contain microtiter plate wells, standards, assay diluent, wash buffer, adhesive plate covers, and/or instructions for carrying out a method of the invention using the kit.

In an aspect of the invention, the kit includes antibodies or antibody fragments which bind specifically to epitopes of kallikrein polypeptides, and means for detecting binding of the antibodies to epitopes associated with a neurodegenerative disease, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains radiolabelled antibody preparations for in vivo imaging.

A kit may be designed to detect the level of polynucleotides encoding kallikrein polypeptides in a sample. Such kits generally comprise oligonucleotide probes or primers, as described herein, that hybridize to polynucleotides encoding kallikrein polypeptides. Such oligonucleotides may be used, for example, within a PCR or hybridization procedure. Additional components that may be present within the kits include second oligonucleotides and/or diagnostic reagents to facilitate detection of polynucleotides encoding kallikrein polypeptides.

The reagents suitable for applying the screening methods of the invention to evaluate compounds may be packaged into convenient kits described herein providing the necessary materials packaged into suitable containers.

Therapeutic Applications

Kallikrein polypeptides may be targets for immunotherapy for neurodegenerative disease. Such immunotherapeutic methods include the use of antibody therapy, in vivo vaccines, and ex vivo immunotherapy approaches.

In one aspect, the invention provides antibodies specific for kallikrein polypeptides that may be used systemically to treat a neurodegenerative disease. Preferably antibodies are used that target the diseased cells/tissues and not surrounding normal cells and tissue. Thus, the invention provides a method of treating a patient susceptible to, or having a primary degenerative demenita that expresses kallikrein polypeptides comprising administering to the patient an effective amount of antibodies that bind specifically to kallikrein polypeptides. Antibodies specific for kallikrein polypeptides may also be used in a method for selectively killing a diseased cell expressing kallikrein polypeptides comprising reacting antibody immunoconjugates or immunotoxins with the cell in an amount sufficient to destroy diseased cells/tissues.

By way of example, unconjugated antibodies specific for kallikrein polypeptides may be introduced into a patient such that the antibodies bind to diseased cells expressing kallikrein polypeptides and mediate destruction thereof of such cells, by mechanisms which may include complement-mediated cytolysis, antibody-dependent cellular cytotoxicity, altering the physiologic function of kallikrein polypeptides and/or the inhibition of ligand binding or signal transduction pathways. In addition to unconjugated antibodies, antibodies specific for kallikrein polypeptides, conjugated to therapeutic agents (e.g. immunoconjugates) may also be used therapeutically to deliver the agents directly to diseased cells expressing kallikrein polypeptides and thereby destroy the cells. Examples of such agents include abrin, ricin A, Pseudomonas exotoxin, or diphtheria toxin, proteins such as tumor necrosis factor, alpha-interferon, beta-interferon, nerve growth factor, platelet derived growth factor, tissue plasminogen activator, and biological response modifiers such as lymphokines, interleukin-1, interleukin-2, interleukin-6, granulocyte macrophage colony stimulating factor, granulocyte colony stimulating factor, or other growth factors.

In the practice of a method of the invention, antibodies specific for kallikrein polypeptides capable of destroying cells expressing kallikrein polypeptides are administered in a therapeutically effective amount to patients whose tumors express or overexpress kallikrein polypeptides. The invention may provide a specific, effective and long-needed treatment for a neurodegenerative disease. The antibody therapy methods of the invention may be combined with other therapies.

Patients may be evaluated for the presence and levels of kallikrein polypeptides expression and overexpression in tisues, preferably using immunohistochemical assessments of tissues, quantitative imaging as described herein, or other techniques capable of reliably indicating the presence and degree of expression of kallikrein polypeptides. Immunohistochemical analysis of biopsies or surgical specimens may be employed for this purpose.

Antibodies specific for kallikrein polypeptides useful in treating a neurodegenerative disease include those that are capable of initiating a potent immune response and those that are capable of direct cytotoxicity. In this regard, the antibodies may elicit disease cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. Potential mechanisms by which such directly cytotoxic antibodies may act include inhibition of cell growth, modulation of cellular differentiation, modulation of angiogenesis, and the induction of apoptosis. The mechanism by which a particular antibody exerts an effect may be evaluated using any number of in vitro assays designed to determine ADCC, antibody-dependent macrophage-mediated cytotoxicity (ADMMC), complement-mediated cell lysis, and others known in the art.

The methods of the invention contemplate the administration of combinations, or “cocktails” of different individual antibodies recognizing epitopes of kallikrein polypeptides. Such cocktails may have certain advantages inasmuch as they contain antibodies that bind to different epitopes and/or exploit different effector mechanisms or combine directly cytotoxic antibodies with antibodies that rely on immune effector functionality. Such antibodies in combination may exhibit synergistic therapeutic effects. In addition, the administration of the antibodies may be combined with other therapeutic agents. The antibodies may be administered in their “naked” or unconjugated form, or may have therapeutic agents conjugated to them.

The antibodies specific for kallikrein polypeptides used in the practice of the method of the invention may be formulated into pharmaceutical compositions comprising a carrier suitable for the desired delivery method. Suitable carriers include any material which when combined with the antibodies retains the function of the antibodies and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

Antibody formulations may be administered via any route capable of delivering the antibodies to the target site. Routes of administration include, but are not limited to, intravenous, intraperitoneal, intramuscular, intratumor, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of neurodegenerative disease and the severity, or stage of the neurodegenerative disease, the binding affinity and half life of the antibodies used, the degree of expression of kallikrein polypeptides in the patient, the extent of circulating kallikrein polypeptide antigens the desired steady-state antibody concentration level, frequency of treatment, and the influence of any therapeutic agents used in combination with a treatment method of the invention.

Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of antibodies necessary to be therapeutically effective in a particular context. Repeated administrations may be required to achieve inhibition or regression. Direct administration of antibodies specific for kallikrein polypeptides is also possible and may have advantages in certain situations.

Patients may be evaluated for kallikrein polypeptides, preferably in CSF or serum, in order to assist in the determination of the most effective dosing regimen and related factors. The assay methods described herein, or similar assays, may be used for quantitating circulating kallikrein polypeptide levels in patients prior to treatment. Such assays may also be used for monitoring throughout therapy, and may be useful to gauge therapeutic success in combination with evaluating other parameters, such as serum kallikrein polypeptides.

The invention further provides vaccines formulated to contain kallikrein polypeptides or fragments thereof. The methods can be practiced by employing kallikrein polypeptides, or fragments thereof, or nucleic acids and recombinant vectors capable of expressing and appropriately presenting the kallikrein immunogens.

By way of example, viral gene delivery systems may be used to deliver nucleic acids encoding kallikrein polypeptides. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding kallikrein polypeptides, or fragments thereof introduced into the patient (e.g., intramuscularly).

Various ex vivo strategies may also be employed. One approach involves the use of cells to present kallikrein antigens to a patient's immune system. For example, autologous dendritic cells which express MHC class I and II, may be pulsed with kallikrein polypeptides, or peptides thereof that are capable of binding to MHC molecules, to thereby stimulate patients' immune systems (See, for example, Tjoa et al., 1996, Prostate 28: 65-69; Murphy et al., 1996, Prostate 29: 371-380).

Anti-idiotypic antibodies specific for kallikrein polypeptides can also be used in therapy as a vaccine for inducing an immune response to cells expressing the polypeptides. The generation of anti-idiotypic antibodies is well known in the art and can readily be adapted to generate anti-idiotypic antibodies that mimic an epitope on a kallikrein polypeptide (see, for example, Wagner et al., 1997, Hybridoma 16: 33-40; Foon et al., 1995, J Clin Invest 96: 334-342; Herlyn et al., 1996, Cancer Immunol Immunother 43: 65-76).

Genetic immunization methods may be utilized to generate prophylactic or therapeutic humoral and cellular immune responses directed against diseased cells expressing one or more kallikrein polypeptides. Constructs comprising DNA encoding kallikrein polypeptides/immunogens and appropriate regulatory sequences may be injected directly into an individual, such that they take-up the construct and express the encoded kallikrein polypeptides/immunogens. The polypeptides/immunogens may be expressed as cell surface proteins or be secreted. Expression of the polypeptides/immunogens results in the generation of prophylactic or therapeutic humoral and cellular immunity. Various prophylactic and therapeutic genetic immunization techniques known in the art may be used.

The invention further provides methods for inhibiting cellular activity (e.g., cell proliferation, activation, or propagation) of a cell expressing kallikrein polypeptides. This method comprises reacting immunoconjugates of the invention (e.g., a heterogeneous or homogenous mixture) with the cell so that the kallikrein polypeptides form a complex with the immunoconjugates. A subject with a neoplastic or preneoplastic condition can be treated when the inhibition of cellular activity results in cell death.

In another aspect, the invention provides methods for selectively inhibiting a cell expressing one or more kallikrein polypeptides by reacting a combination of the immunoconjugates of the invention with the cell in an amount sufficient to inhibit the cell. Amounts include those that are sufficient to kill the cell or sufficient to inhibit cell growth or proliferation.

Vectors derived from retroviruses, adenovirus, herpes or vaccinia viruses, or from various bacterial plasmids, may be used to deliver nucleic acids encoding kallikrein polypeptides to a targeted organ, tissue, or cell population. Methods well known to those skilled in the art may be used to construct recombinant vectors that will express antisense nucleic acid molecules for kallikrein polypeptides. (See, for example, the techniques described in Sambrook et al (supra) and Ausubel et al (supra)).

Genes encoding kallikrein polypeptides can be turned off by transfecting a cell or tissue with vectors that express high levels of desired kallikrein polypeptide-encoding fragments. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of genes encoding kallikrein polypeptides, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding one or more kallikrein polypeptides.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

Kallikrein polypeptides and polynucleotides encoding the polypeptides, and fragments thereof, or agents identified using a method of the invention may be used in the treatment of a neurodegenerative disease in a subject. The kallikrein polypeptides, polynucleotides, and agents may be formulated into compositions for administration to subjects suffering from a neurodegenerative disease. Therefore, the present invention also relates to a composition comprising one or more kallikrein polypeptides, or polynucleotides encoding the polypeptides, or a fragment thereof, or an agent identified using a method of the invention, and a pharmaceutically acceptable carrier, excipient or diluent. A method for treating or preventing a neurodegenerative disease in a subject is also provided comprising administering to a patient in need thereof, kallikrein polypeptides, or polynucleotides encoding the polypeptides, an agent identified in accordance with a method of the invention, or a composition of the invention.

The active substance may be administered in a convenient manner such as by injection (subcutaneous, intravenous, etc.), oral administration, inhalation, transdermal application, or. rectal administration. Depending on the route of administration, the active substance may be coated in a material to protect the substance from the action of enzymes, acids and other natural conditions that may inactivate the substance. An active substance may be administered to the CNS parenterally or intraperitoneally. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). The compositions, may include, for example, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment. The compositions of the invention may be administered concurrently, separately, or sequentially with other therapeutic agents or therapies. The therapeutic activity of antibodies specific for kallikrein polypeptides, compositions, and compounds identified using a method of the invention and may be evaluated in vivo using a suitable animal model, for example, the animal model described in U.S. patent application No. 20030101467 published May 29, 2003.

The following non-limiting examples are illustrative of the present invention:

### EXAMPLE 1

Various kallikreins were quantitated in CSF of 20 patients with Alzheimer's disease [AD], 16 patients with frontotemporal dementia [FTD] and 15 controls. The levels of various kallikreins were correlated with the presence of AD or FTD. Among all kallikreins measured, detectable levels in CSF were identified for kallikreins hK6, hK7 and hK10. Other tested kallikreins [hK5, hK8, hK11 and hK13] were unmeasurable. The most notable differences between kallikrein levels in CSF and the three groups of subjects were seen between controls and FTD patients for hK6 [decrease in FTD; p=0.017], controls and FTD patients for hK7 [decrease in FTD; p<0.001] and controls and AD patients for hK7 [decrease in AD; p=0.019]. Also, significant differences were seen between FTD patients or control subjects and patients with AD patients for hK10 [increase in AD; p≦0.02]. Approximately half of the AD patients had CSF hK10 levels that were higher than all patients with FTD except one and all control subjects except two. Various kallikrein concentrations in CSF were correlated, the strongest correlation was seen between hK6 and hK7 [rs=0.58]. A statistically significant association was observed between decreasing hK7 concentration in CSF and possession of one or two ApoE4 alleles (p=0.014).

In conclusion, significant alterations of hK6, hK7 and hK10 concentrations were observed in CSF of patients with AD and FTD. Notably, all 3 kallikreins (hK6, hK7 and hK10) are decreased in CSF of FTD patients and hK10 is increased in CSF of AD patients, in comparison to control subjects.

Materials and Methods

Subjects

Included in the study were 20 patients with probable AD (age range 57-75 years), 16 patients with FTD (age range 48-77 years), and 15 controls (age range 54-77 years). Their characteristics are summarised in Table 1.

All patients included in the study had a clinical diagnosis of FTD and AD and were consecutively recruited from prospective longitudinal studies of patients with dementia or psychiatric disease. Clinical diagnoses were established and CSF sampling was performed. Neurochemical analyses were performed at the Institute of Clinical Neuroscience, Sahlgrenska University Hospital, Mölndal, Sweden. Excluded were patients with unspecified dementia (e.g. mixed dementia), psychiatric disease (e.g. schizophrenia), chronic alcoholism, distinct non-degenerative neurological disease (e.g. normal pressure hydrocephalus), a history of severe head injury, infections in the CNS or systemic diseases (e.g. malignant tumours) or secondary causes (e.g. hypothyroidism) of dementia according to the Diagnostic and Statistical Manual of Mental Disorders (American Psychiatric Association, 1987) or biochemical criteria. Excluded were also patients with large cerebral infarcts and/or multiple lacunas. All included patients underwent a thorough clinical investigation, including medical history, physical, neurological and psychiatric examinations, screening blood laboratory tests (relevant laboratory tests to exclude other causes of dementia e.g. hypothyroidism), routine analysis of the CSF (e.g. cytology), ECG, chest X-ray, EEG, computerised tomography (CT) or magnetic resonance imaging (MRI) of the brain, investigation of the regional cerebral blood flow (rCBF) using either single photon emission computerized tomography (SPECT) or 133Xenon inhalation technique (Cortexplorer).

FTD was diagnosed according to the Lund/Manchester criteria (core diagnostic features; (Neary et al., 1998) as previously described. None of the FTD patients had signs of infarcts, and only mild punctata white matter changes were found in two FTD patients.

The diagnosis of AD was made by exclusion, in accordance with the NINCDS-ADRDA. criteria (McKhann et al., 1984). The AD patients were divided into one group with probable AD and another with possible AD, as defined by the NINCDS-ADRDA criteria.

All the clinical diagnoses were made by physicians without knowledge of the results of the biochemical analyses and vice versa. None of the patients were currently treated for dementia (e.g. with cholinesterase inhibitors).

In the demented patients, the degree of dementia was evaluated using the mini-mental state examination score (MMSE) (Folstein et al., 1975).

The control group consisted of individuals without history, symptoms or signs of psychiatric or neurological disease, malignant disease or systemic disorders (e.g. rheumatoid arthritis, infectious disease). MMSE was used to evaluate their cognitive status, and those with scores below 28 were excluded.

The Ethics Committee of Göteborg University approved the study. All the patients (or their next of kin) and controls gave their informed consent for participating in the study, which was conducted in accordance with the provisions of the Helsinki Declaration.

CSF Collection and Analysis

Lumbar puncture was performed in all patients and controls at the L3/L4 or L4/L5 interspace. The first 12 mL of CSF were collected in polypropylene tubes and gently mixed to avoid gradient effects (Blennow et al., 1993). All CSF samples with more than 500 erythrocytes per μL were excluded. The CSF samples were centrifuged at 2000×g for 10 min to eliminate cells and other insoluble material. Aliquots were then stored at −80° C. until biochemical analysis.

CSF samples were diluted as necessary in a 60 g/L bovine serum albumin solution and then analyzed for the following kallikreins, using immunofluorometric procedures developed in-house: hK5, hK6, hK7, hK8, hK10, hK11 and hK13. The assays for human kallikreins 5, 6, 8, 10, 11 and 13 have been published elsewhere (Diamandis et al., 2000b; Yousef et al., in press a; Kishi et al., 2003; Luo et al., 2001; Diamandis et al., 2002; Kapadia et al., 2003). The method for quantifying hK7 is similar to the one published for hK8 but specific polyclonal mouse and rabbit antibodies against hK7 were used. For all assays, the cross-reactivity was tested with other members of the kallikrein family. None of them cross-reacted with other kallikreins to any significant degree. All assays were calibrated with recombinant proteins in yeast and mammalian expression systems, as previously described (Diamandis et al., 2000b; Yousef et al., in press a; Kishi et al., 2003; Luo et al., 2001; Diamandis et al., 2002; Kapadia et al., 2003). The detection limits of these assays (in μg/L) were as follows: hK5 (0.1), hK6 (0.5), hK7 (0.1), hK8 (0.2), hK10 (0.05), hK11 (0.1) and hK13 (0.05). The within-run and day-to-day precision of all assays was less than 10%. All samples were analyzed at least in duplicate. Samples with levels outside the measurement range of the assay were diluted and re-analyzed. All analyses were performed in-blind and the code was broken by the Biostatistician after all measurements had been completed and data entered into the database.

Determination of ApoE Isoforms

Depending on the sample material available, determination of ApoE isoforms was performed either by isoelectric focusing (IEF) and Western blotting with minor modifications (Kane and Gowland, 1986; Skoog et al., 1998), or by polymerase chain-reaction and DNA hybridization using the Innolipa ApoE kit [Innogenetics, Ghent, Belgium].

Results

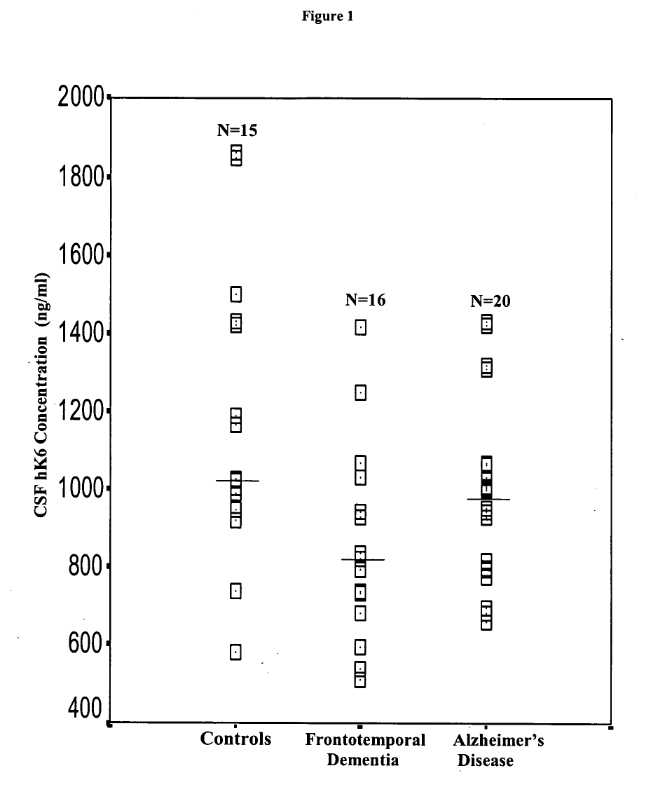

Table 2 shows the mean, standard error, median and range of kallikreins hK6, hK7 and hK10 in the three groups of subjects (controls, N=15; FTD, N=16; and AD, N=20). The concentrations of kallikreins hK5, hK8, hK11 and hK13 in cerebrospinal fluid of these subjects was not measurable with the assays used and these kallikreins were excluded from further statistical analysis. The data are further graphically presented in FIGS. 1, 2 and 3.

For hK6, a statistically significant difference between controls and FTD patients was found (p=0.017 by the Mann-Whitney test) but not between any other groups. Levels were lower in FTD.

For hK7, a highly significant decrease of this enzyme was found in CSF of patients with FTD versus controls (30% decrease in medians; p<0.001) and between AD and controls (12% decrease in medians; p=0.019). The difference between medians of FTD patients and AD patients was of marginal statistical significance (p=0.046).

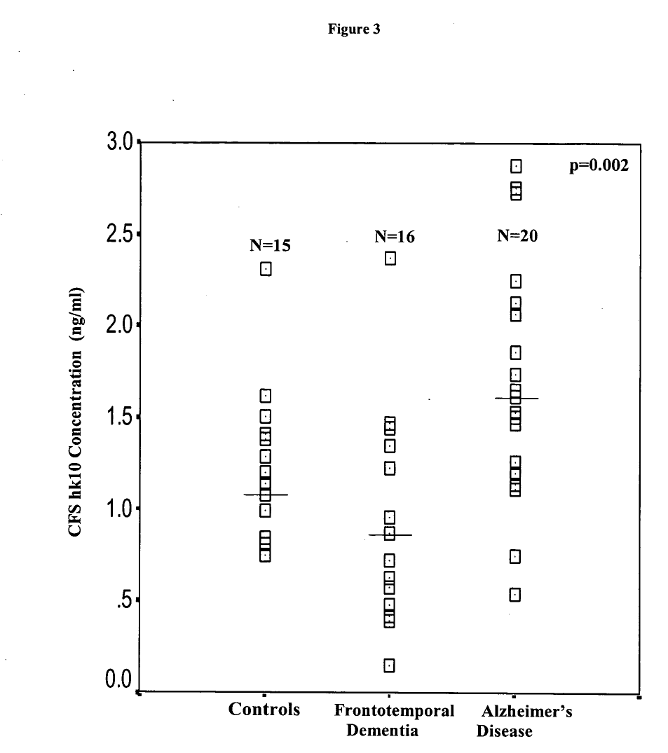

For hK10, there was no statistically significant difference between controls and FTD patients (p=0.10) but a highly significant difference between FTD or control subjects and patients with AD (p=0.001 and 0.016, respectively). Notably, approximately half of the AD patients had CSF hK10 levels which were higher than all patients with FTD, except one and all control subjects except two (FIG. 3).

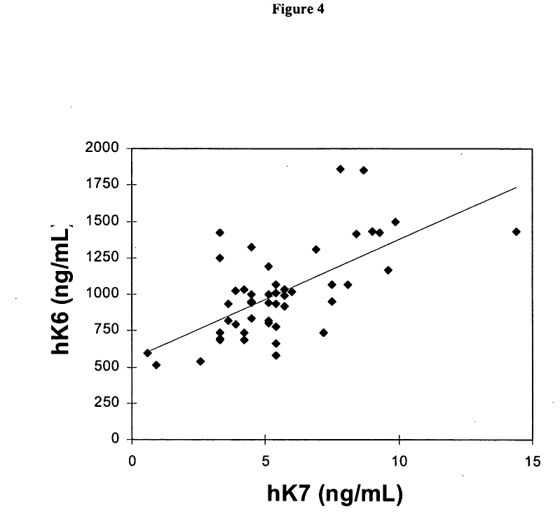

Table 3 shows that the Spearman correlation analysis between levels of the three kallikreins and age of patients, duration of the dementia and MMSE score. For this analysis, only the patient groups were included. Statistically significant correlations were seen between age and hK10 (positive correlation), duration of the disease and MMSE score (negative correlation) and between kallikreins hK6 and hK7, hK6 and hK10 and hK7 and hK10 (all positive correlations). The strongest correlation was seen between hK6 and hK7 concentration (rs=0.58). This correlation was also demonstrated when all subjects in this study were included (FIG. 4; N=51).

Table 4 summarizes the associations between levels of hK6, hK7 and hK10 in CSF and ApoE4 genotypes. Statistically significant differences with a consistent trend were seen only with hK7; possession of one or two ApoE4 alleles was associated with progressively lower levels of hK7 in CSF.

Discussion

Despite intense investigations on the genetics and biology of neurodegenerative disorders, including AD and FTD, their pathogenesis is still elusive. Recently, the contribution of proteases involved in amyloid precursor protein processing to the pathogenesis of AD has been described (Hardy and Selkoe, 2002; Selkoe, 2001; Selkoe, 2002). Therapeutic strategies that target these enzymes are currently in clinical trials (Yamada and Toshitaka, 2002; Citron, 2002; Morelli et al., 2002). It is likely that the pathogenesis of these disorders is associated with as yet unidentified proteolytic pathways operating in the CNS (Yousef et al., 2003).

Recently, the complete organization of the human tissue kallikrein gene locus on chromosome 19q13.4 was described (Diamandis et al., 2000a; Yousef et al., 2001). Among all serine proteases within the human genome, this cluster is, by far, the largest (Yousef et al., in press b). The genomic organization of this family and the pattern of its expression have prompted speculation that it may represent a new enzymatic cascade pathway which operates in various tissues (Yousef and Diamandis, 2002). Some kallikreins have been found at relatively high levels in CSF. A few studies have already associated AD with levels of kallikrein 11 and kalllikrein 6 in either brain tissues or CSF (Diamandis et al., 2000c; Zarghooni et al., 2002; Mitsui et al., 2002; Little et al., 1997; Scarisbrick et al., 2001; Scarisbrick et al., 2002).

The association between CSF kallikrein concentration in AD or FTD was examined. Using quantitative methodologies, at least 3 kallikreins, hK6, hK7 and hK10, can be reliably quantified in CSF. hK6 concentration is significantly reduced in CSF of patients with FTD, in comparison to either control subjects or AD patients. Furthermore, significant decreases of hK7 were seen in patients with FTD, in comparison to either control subjects or patients with AD. Similar decreases in CSF of FTD patients were also seen with hK10. Thus, all 3 measurable kallikreins in CSF (hK6, hK7 and hK10 ) are decreased in patients with FTD, in comparison to control subjects.

No data have as yet been published on the levels of hK10 in CSF of patients with neurodegenerative disorders. Highly significant elevations of hK10 were reported in approximately half of AD patients, in comparison to either control subjects or patients with FTD.

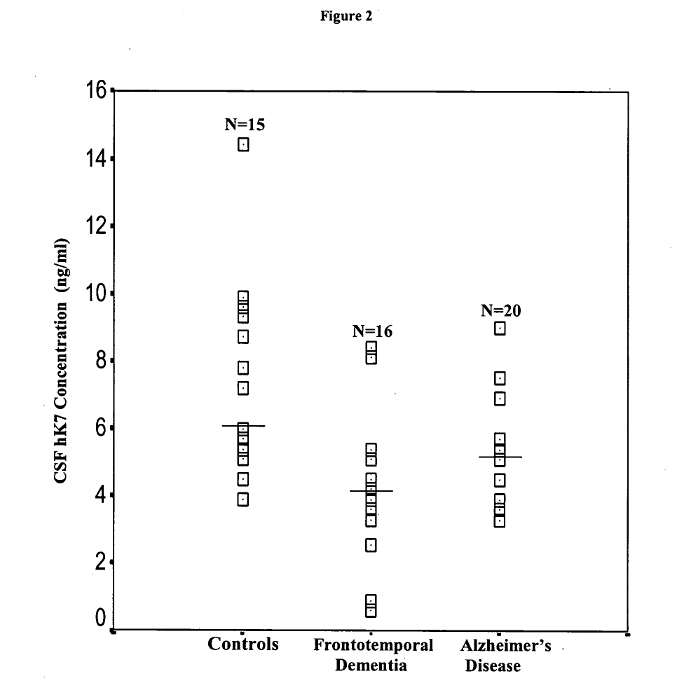

hK6, hK67 and hK10 concentrations in CSF are correlated with each other, the strongest correlation being between hK6 and hK7. Furthermore, an association was established between decreasing levels of CSF hK7 (which are also lower in AD patients than controls; Table 2 and FIG. 2) and possession of either one or two ApoE4 alleles (Table 4). It is known that this allele is associated with an increased risk of AD (Evans et al., 2003). Lower levels of hK6, hK7 and hK10 in CSF may represent new risk factors for FTD while lower levels of hK7 and increased levels of hK10 in CSF may represent new risk factors for AD.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

TABLE 1
Characteristics of the patient population and controls

| | | | | | Duration | Degree of |
|---|---|---|---|---|---|---|
| | | | | Age at | of | dementia |
| | | Age | Gender | onset | dementia | (MMSE |
| Diagnosis | N | (years) | (F:M) | (years) | (years) | Score) |
| FTD | 16 | 63.6 ± 9.0 | 12:4 | 58.6 ± 9.2 | 4.9 ± 3.8 | 17.2 ± 7.6 |
| AD | 20 | 68.2 ± 5.5 | 12:8 | 64.4 ± 6.2 | 3.8 ± 3.1 | 18.4 ± 3.1 |
| Controls | 15 | 67.9 ± 5.6 | 9:6 | — | — | 29.6 ± 0.5 |

All values are expressed as means ± SD.
The following abbreviations are used:
FTD = frontotemporal dementia,
AD = Alzheimer's disease,
N = number of individuals;
F = female,
M = male.

TABLE 2
Descriptive statistics for hk6, hk7 and hk10 protein levels in CFS
of controls, frontotemporal dementia and Alzheimer's
disease patients.

| | | Standard | | | |
|---|---|---|---|---|---|
| | Mean | Error | Median | Range | p value* |
| hK6 (ng/ml) | | | | | |
| Controls | 1176 | 96 | 1026 | 582-1860 | 0.017a |
| (N = 15) | | | | | |
| Frontotemporal | 872 | 63 | 828 | 510-1416 | 0.21b |
| Dementia | | | | | |
| (N = 16) | | | | | |
| Alzheimer's | 985 | 53 | 975 | 660-1428 | 0.12c |
| disease | | | | | |
| (N = 20) | | | | | |
| hK7 (ng/ml) | | | | | |
| Controls | 7.18 | 0.73 | 6.00 | 3.90-14.4 | <0.001a |
| (N = 15) | | | | | |
| Frontotemporal | 4.17 | 0.52 | 4.20 | 0.60-8.40 | 0.046b |
| Dementia | | | | | |
| (N = 16) | | | | | |
| Alzheimer's | 5.26 | 0.34 | 5.25 | 3.30-9.00 | 0.019c |
| disease | | | | | |
| (N = 20) | | | | | |
| hK10 (ng/ml) | | | | | |
| Controls | 1.20 | 0.11 | 1.14 | 0.75-2.31 | 0.10a |
| (N = 15) | | | | | |
| Frontotemporal | 0.93 | 0.14 | 0.79 | 0.15-2.37 | 0.001b |
| Dementia | | | | | |
| (N = 16) | | | | | |
| Alzheimer's | 1.66 | 0.14 | 1.57 | 0.54-2.88 | 0.016c |
| disease | | | | | |
| (N = 20) | | | | | |

*Calculated by the Mann-Whitney test
aControls vs Frontotemporal Dementia
bFrontotemporal Dementia vs Alzheimer's Disease
cAlzheimer's Disease vs Controls

TABLE 3
Correlations between the studied variables in frontotemporal
dementia or Alzheimer's disease patients

| | | Duration | MMSE | hK6 | hK7 | hK10 |
|---|---|---|---|---|---|---|
| Age | rs | −0.067 | 0.079 | 0.256 | 0.166 | 0.387 |
| | p | 0.698 | 0.658 | 0.133 | 0.333 | 0.020 |
| | N | 36 | 34 | 36 | 36 | 36 |
| Duration | rs | | −0.406 | −0.166 | 0.013 | 0.159 |
| | p | | 0.017 | 0.333 | 0.939 | 0.355 |
| | N | | 34 | 36 | 36 | 36 |
| MMSE | rs | | | 0.033 | 0.056 | −0.152 |
| | p | | | 0.854 | 0.753 | 0.390 |
| | N | | | 34 | 34 | 34 |
| hK6 | rs | | | | 0.577 | 0.420 |
| | p | | | | 0.000 | 0.011 |
| | N | | | | 36 | 36 |
| hK7 | rs | | | | | 0.430 |
| | p | | | | | 0.009 |
| | N | | | | | 36 |

TABLE 4
Relationships between hk6, hk7, hk10 protein levels (ng/ml)
in CSF and Apo E genotypes

| | | Standard | | | |
|---|---|---|---|---|---|
| | Mean | Error | Median | Range | p value* |
| hK6 (ng/ml) | | | | | |
| ApoE | | | | | |
| 0 E4 Allele | 1160 | 69.3 | 1068 | 594-1860 | 0.019 |
| (N = 22) | | | | | |
| 1 E4 Allele | 879 | 41.9 | 936 | 582-1032 | |
| (N = 12) | | | | | |
| 2 E4 Alleles | 950 | 95.7 | 930 | 540-1422 | |
| (N = 9) | | | | | |
| hK7 (ng/ml) | | | | | |
| ApoE | | | | | |
| 0 E4 Allele | 6.47 | 0.61 | 5.70 | 0.60-14.4 | 0.014 |
| (N = 22) | | | | | |
| 1 E4 Allele | 4.97 | 0.33 | 5.25 | 3.30-7.50 | |
| (N = 12) | | | | | |
| 2 E4 Alleles | 4.28 | 0.47 | 4.20 | 2.55-7.50 | |
| (N = 9) | | | | | |
| hK10 (ng/ml) | | | | | |
| ApoE | | | | | |
| 0 E4 Allele | 1.18 | 0.098 | 1.21 | 0.39-2.37 | 0.73 |
| (N = 22) | | | | | |
| 1 E4 Allele | 1.37 | 0.17 | 1.20 | 0.54-2.73 | |
| (N = 12) | | | | | |
| 2 E4 Alleles | 1.42 | 0.31 | 1.62 | 0.15-2.88 | |
| (N = 9) | | | | | |

*Calculated by Kruskal Wallis Test

TABLE 5

| | Kallikrein | |
|---|---|---|
| Kallikrein | Nucleic Acid | GenBank |
| Polypeptide | Designation | Accession No. |
| Kallikrein 6 | KLK6 | AF013988, AF149289, U62801 |
| Kallikrein 7 | KLK7 | L33404, AF166330 |
| Kallikrein 10 | KLK10 | AF055481, NM_002776 |
| Kallikrein 11 | KLK11 | AB012917, AF164623 |

Full Citations for References Referred to in the Specification
1. American Psychiatric Association. Diagnostic and Statistical Manual of Mental Disorders, 3rd ed.—Revised, Washington D.C., Cambridge University Press, 1987.
2. Black M H, Magklara A, Obiezu C, Levesque M A, Sutherland D J A, Tindall D J, Young C Y F, Sauter E R, Diamandis E P. Expression of a prostate-associated protein, human glandular kallikrein (hK2), in breast tumors and in normal breast secretions. Br J Cancer 2000;82:361-367.
3. Blennow K, Wallin A. Clinical heterogeneity of probable Alzheimer's disease. J Geriatr Psychiatry Neurol 1992;5:106-113.
4. Blennow K, Fredman P, Wallin A, Gottfries C-G, Langstrom L, Svennerholm L.

Protein analysis in cerebrospinal fluid: I. Influence of concentration gradients for proteins on cerebrospinal fluid/serum albumin ratio. Eur Neurol 1993;33:126-128.
5. Citron M. Emerging Alzheimer's disease therapies: inhibition of beta-secretase. Neurobiol Aging 2002;23:1017-1022.
6. Diamandis E P, Yousef G M, Luo L Y, Magklara A, Obiezu C V. The human kallikrein gene family—implications in carcinogenesis. Trends Endocrinol Metab 2000; 11:54-60.
7. Diamandis E P, Yousef G M, Soosaipillai A R, Grass L, Porter A, Little S, Sotiropoulou G. Immunofluorometric assay of human kallikrein 6 (zyme/protease M/neurosin) and preliminary clinical applications. Clin Biochem 2000;33:369-375.
8. Diamandis E P, Yousef G M, Petraki C, Soosaipillai A R. Human kallikrein 6 as a biomarker of Alzheimer's disease. Clin Biochem 2000;33:663-667.
9. Diamandis E P, Yousef G M, Clements J, Ashworth L K, Yoshida S, Egelrud T, Nelson P S, Shiosaka S, Little S, Lilja H, Stenman U H, Rittenhouse H G, Wain H. New nomenclature for the human tissue kallikrein gene family. Clin Chem 2000;46:1855-1858.
10. Diamandis E P, Okui A, Mitsui S, Luo L Y, Soosaipillai A, Grass L, Nakamura T, Howarth D J, Yamaguchi N. Human kallikrein 11: a new biomarker of prostate and ovarian carcinoma. Cancer Res 2002;62:295-300.
11. Evans D A, Bennett D A, Wilson R S, Bienias J L, Morris M C, Scherr P A, Hebert L E, Aggarwal N, Beckett L A, Joglekar R, Berry-Kravis E, Schneider J. Incidence of Alzheimer disease in a biracial urban cummunity: relation to apolipoprotein E allele status. Arch Neurol 2003;60:185-189.
12. Folstein M, Folstein S, McHugh P. “Mini-mental State” A practical method for grading the cognitive state of patients for the clinician. J Psychiatr Res 1975;12:189-198.
13. Hardy J, Selkoe D J The amyloid hypothesis of Alzheimer's disease: progress and problems on the road to therapeutics. Science 2002;297:353-356.
14. Kane J W, Gowland E. A method for the identification of apolipoprotein E isoforms employing chemical precipitation and flat bed isoelectric focusing in agarose. Ann Clin Biochem 1986;23:509-513.
15. Kapadia C, Chang A, Sotiropoulou G, Yousef G M, Grass L, Soosaipillai A, Xing X, Howarth D H, Diamandis E P. Human kallikrein 13: production and purification of recombinant protein and monoclonal and polyclonal antibodies, and development of a sensitive and specific immunofluorometric assay. Clin Chem 2003;49:77-86.
16. Kishi T, Grass L, Soosaipillai A, Shimizu-Okabe C, Diamandis E P. Human kallikrein 8: immunoassay development and identification in tissue extracts and biological fluids. Clin Chem 2003;49:87-96.
17. Little S P, Dixon E P, Norris F, Buckley W, Becker G W, Johnson M, Dobbins J R, Wyrick T, Miller J R, MacKellar W, Hepburn D, Corvalan J, McClure D, Liu X, Stephenson D, Clemens J, Johnstone E M. Zyme, a novel and potentially amyloidogenic enzyme cDNA isolated from Alzheimer's disease brain. J Biol Chem 1997;272:25135-25142.
18. Luo L Y, Grass L, Howarth D J, Thibault P, Ong H, Diamandis E P. Immunofluorometric assay of human kallikrein 10 and its identification in biological fluids and tissues. Clin Chem 2001;47:237-246.
19. McKhann G, Drachman D, Folstein M, Katzman R, Price D, Stadlan E M. Clinical diagnosis of Alzheimer's disease: report on the NINCDS-ADRDA Work group under the auspices of department of health and human services task force on Alzheimer's disease. Neurology 1984;34:939-944.
20. Mitsui S, Okui A, Uemura H, Mizuno T, Yamada T, Yamamura Y, Yamaguchi N. Decreased cerebrospinal fluid levels of neurosin (KLK6), an aging-related protease, as a possible new risk factor for Alzheimer's disease. Ann NY Acad Sci 2002;977:216-233.
21. Mitsui S, Yamada T, Okui A, Kominami K, Uemura H, Yamaguchi N. A novel isoform of a kallikrein-like protease, TLSP/hippostasin, (PRSS20), is expressed in the human brain and prostate. Biochem Biophys Res Commun 2000;272:205-211.
22. Morelli L, Llovera R, Ibendahl S, Castano E M. The degradation of amyloid beta as a therapeutic strategy in Alzheimer's disease and cerebrovascular amyloidoses.

Neurochem Res 2002;27:1387-1399.
23. Neary D, Snowden J S, Gustafson L, Passant U, Stuss D, Black S, Freedman M, Kertesz A, Robert P H, Albert M, Boone K, Miller B L, Cummings J, Benson D F. Frontotemporal lobar degeneration: a consensus on clinical diagnostic criteria. Neurology 1998;5 1: 1546-1554.
24. Ogawa K, Yamada T, Tsujioka Y, Taguchi J, Takahashi M, Tsuboi Y, Fujino Y, Nakajima M, Yamamoto T, Akatsu H, Mitsui S, Yamaguchi N. Localization of a novel trypsin-like serine protease, neurosin, in brain tissues of Alzheimer's disease and Parkinson's disease. Phychiatry Clin Neurosci 2000;54:419-426.
25. Pasquier F. Neuropsychological features and cognitive assessment in frontotemporal dementia; in Pasquier F, Lebert F Scheltens P, Editors: Frontotemporal dementia. Dordrecht, ICG Publications, 1996, 8, pp. 49-70.
26. Petraki C D, Karavana V N, Skoufogiannis P T, Little S P, Howarth D J C, Yousef G M, Diamandis E P. The spectrum of human kallikrein 6 (zyme/Protease M/neurosin) expression in human tissues as assessed by immunohistochemistry. J Histochem Cytochem 2001;49:1431-1441.
27. Rideout H J, Zang E, Yeasmin M, Gordon R, Jabado O, Parks D S, Stefanis L. Inhibitors of trypsin-like serine proteases prevent DNA damage-induced neuronal death by acting upstream of the mitochondrial checkpoint and of p53 induction. Neurosccience 2001 ;107:339-352.
28. Selkoe D J. Alzheimer's disease: genes, proteins and therapy. Physiol Rev 2001;81:741-766.
29. Selkoe D J. Alzheimer's disease is a synaptic failure. Science 2002;298:789-791.
30. Scarisbrick I A, Isackson P J, Ciric B, Windebank A J, Rodriguez M. MSP, a trypsin-like protease, is abundantly expressed in the human nervous system. J Comp Neurol 2001; 431:347-361.
31. Scarisbrick I A, Blaber S I, Lucchinetti C F, Genain C P, Blaber M, Rodriguez M. Activity of a newly identified serine protease in CNS demyelination. Brain 2002;125:1283-1296.
32. Shimizu-Okabe C, Yousef G M, Diamandis E P, Yoshida S, Shiosaka S, Fahnestock M. Expression of the kallikrein gene family in normal and Alzheimer's disease brain. Neuroreport 2001;12:2747-2751.
33. Sjögren H. Twenty-four cases of Alzheimer's disease. Acta Med Scand 1950; 245: 225-233.
34. Sjögren M, Rosengren L, Minthon L, Davidsson P, Blennow K, Wallin A. Cytoskeleton proteins in CSF distinguish frontotemporal dementia from AD. Neurology 2000;54: 1960-1964.
35. Sjogren M, Wallin A. Pathophysiological aspects of frontotemporal dementia—emphasis on cytoskeleton proteins and autoimmunity. Mech Ageing Dev 2001;122:1923-1935.
36. Skoog I, Hesse C, Aevarsson O, Landahl S, Wahlstrom J, Fredman P, Blennow K. A population study of apoE genotype at the age of 85: relation to dementia, cerebrovascular disease, and mortality. J Neurol Neurosurg Psychiatry 1998;64:37-43.
37. Sourander P, Sjögren H. The concept of Alzheimer's disease and its clinical implications; in
38. Wolstenholme GEWO'Connors M, Editors: Alzheimer's disease. London, Ciba Foundation Symposium, 1970, pp. 11-36.
39. Yamada K, Toshitaka N. Therapeutic approaches to the treatment of Alzheimer's disease. Drugs Today 2002;38:631-637.
40. Yoshida S, Taniguchi M, Suemoto T, Oka T, He X, Shiosaka S. cDNA cloning and expression of a novel serine protease, TLSP. Biochim Biophys Acta 1998;1399:225-228.
41. Yoshida S, Shiosaka S. Plasticity-related serine proteases in the brain (review). Int J Mol Med 1999;3:405-409.
42. Yousef G M, Diamandis E P. The new human tissue kallikrein gene family: structure, function and association to disease. Endocr Rev 2001;22:184-204.
43. Yousef G M, Luo L-Y, Scherer S W, Sotiropoulou G, Diamandis E P. Molecular characterization of zyme/protease M/neurosin (PRSS( ), a hormonally regulated kallikrein-like serine protease. Genomics 1999;62:251-259.
44. Yousef G M, Scorilas A, Magklara A, Soosaipillai A, Diamandis E P. The KLK7)PRSS6) gene, encoding for the stratum corneum chymotryptic enzyme is a new member of the human kallikrein gene family-genomic characterization, mapping, tissue expression and hormonal regulation. Gene 2000a;254:119-128.
45. Yousef G M, Scorilas A, Diamandis E P. Genomic organization, mapping, tissue expression and hormonal regulation of trypsin-like serine protease (TLSP PRSS20), a new member of the human kallikrein gene family. Genomics 2000b;63:88-96.
46. Yousef G M, Diamandis E P. Human tissue kallikrein s: a new enzymatic cascade pathway? Biol Chem 2002;383:1045-1057.
47. Yousef G M, Kishi T, Diamandis E P. Role of kallikrein enzymes in the central nervous system. Clin Chim Acta 2003;329:1-8.
48. Yousef G M, Grass L, Polymeris M-E, Soosaipillai A, Chan P-C, Scorilas A, Borgono C, Harbeck N, Schmalfeldt B, Dorn J, Schmitt M, Diamandis E P. Human kallikrein 5 (hK5): a novel serum biomarker for breast and ovarian cancer. Cancer Res, in press [a].
49. Yousef G M, Kopolovic A D, Elliott M B, Diamandis E P. Genomic overview of serine proteases. Biochem Biophys Res Commun, in press [b].
50. Zarghooni M, Soosaipilla A, Grass L, Scorilas A, Mirazimi N, Diamandis E P. Decreased concentration of human kallikrein 6 in brain extracts of Alzheimer's disease patients. Clin Biochem 2002;35:225-231.

1. A method for detecting kallikrein polypeptides or polynucleotides encoding kallikrein polypeptides that are associated with a neurodegenerative disease in a patient comprising:
(a) taking a sample from a patient;
(b) detecting or identifying in the sample one or more kallikrein polypeptides or polynucleotides encoding the kallikrein polypeptides, wherein the kallikrein polypeptides comprise or are selected from the group consisting of kallikrein 7 and kallikrein 10 and
(c) comparing the detected amount with an amount detected for a standard.

2. A method for detecting kallikrein polypeptides associated with a neurodegenerative disease in a patient comprising:
(a) obtaining a sample from a patient;
(b) detecting in the sample one or more kallikrein polypeptides, wherein the kallikrein polypeptides comprise or are selected from the group consisting of kallikrein 7 and kallikrein 10; and
(c) comparing the detected amounts with amounts detected for a standard.

3. A method for diagnosing and monitoring a neurodegenerative disease in a subject comprising detecting in a sample from the subject one or more kallikrein polypeptides, wherein the kallikrein polypeptides comprise or are selected from the group consisting of kallikrein 7 and kallikrein 10.

4. (currently amended) A method as claimed in claim 1, wherein the kallikrein polypeptides are detected using antibodies that bind to the kallikrein polypeptides or parts thereof

5. A method of detecting a neurodegenerative disease in a patient, the method comprising comparing:
(a) levels of one or more kallikrein polypeptides in a sample from the patient, wherein the kallikrein polypeptides comprise or are selected from the group consisting of kallikrein 7 and kallikrein 10; and
(b) normal levels of expression of the kallikrein polypeptides in a control sample, wherein a significant difference in levels of kallikrein polypeptides, relative to the corresponding normal levels, is indicative of a neurodegenerative disease.

6. A method of assessing whether a patient is afflicted with or has a pre-disposition for Alzheimer's Disease, the method comprising comparing:
(a) levels of one or both kallikrein polypeptides in a sample from the patient wherein the kallikrein polypeptides comprise kallikrein 7 and kallikrein 10 associated with Alzheimer's Disease; and
(b) normal levels of the kallikrein polypeptides, in samples of the same type obtained from control patients not afflicted with Alzheimer's Disease, wherein significantly lower levels of kallikrein 7 and/or higher levels of kallikrein 10, relative to the corresponding normal levels of the kallikrein polypeptides, is an indication that the patient is afflicted with Alzheimer's Disease.

7. A method of assessing whether a patient is afflicted with or has a pre-disposition for frontotemporal dementia, the method comprising comparing:
(a) levels of one or both kallikrein polypeptides in a sample from the patient wherein the kallikrein polypeptides comprise kallikrein 7 and kallikrein 10 associated with frontotemporal dementia; and
(b) normal levels of the kallikrein polypeptides, in samples of the same type obtained from control patients not afflicted with frontotemporal dementia, wherein significantly lower levels of kallikrein 7 and/or lower levels of kallikrein 10, relative to the corresponding normal levels of the kallikrein polypeptides, is an indication that the patient is afflicted with frontotemporal dementia.

8. A method for monitoring the progression of a neurodegenerative disease in a patient, the method comprising: (a) detecting in a sample from the patient at a first time point, one or more kallikrein polypeptides, wherein the kallikrein polypeptides comprise or are selected from the group consisting of kallikrein 7 and kallikrein 10; (b) repeating step (a) at a subsequent point in time; and (c) comparing levels detected in steps (a) and (b), and thereby monitoring the progression of a neurodegenerative disease.

9. A method for diagnosing and monitoring a neurodegenerative disease in a sample from a subject comprising isolating polynucleotides from the sample, and detecting in the sample one or more polynucleotides encoding kallikrein polypeptides, wherein the kallikrein polypeptides comprise or are selected from the group consisting of kallikrein 7 and kallikrein 10.

10. A method as claimed in claim 9 wherein significant differences in the levels of the polynucleotides in the sample compared to a control is indicative of disease, disease stage, stage, and/or prognosis.

11. A method for determining the presence or absence of a neurodegenerative disease in a subject comprising: (a) contacting a sample obtained from the subject with oligonucleotides that hybridize to polynucleotides encoding one or more kallikrein polypeptides, wherein the kallikrein polypeptides comprise or are selected from the group consisting of kallikrein 7 and kallikrein 10; and (b) detecting in the sample levels of nucleic acids that hybridize to the polynucleotides encoding kallikrein polypeptides relative to a predetermined cut-off value, and therefrom determining the presence or absence of a neurodegenerative disease in the subject.

12. A method as claimed in claim 11, wherein the nucleic acids are mRNA and the levels of nucleic acids are detected by polymerase chain reaction.

13. A method as claimed in claim 11 wherein the nucleic acids are mRNA and the amounts of mRNA are detected using a hybridization technique, employing oligonucleotide probes that hybridize to kallikrein polypeptides.

14. A method for assessing the potential efficacy of a test agent for inhibiting a neurodegenerative disease in a patient, the method comprising comparing: (a) levels of one or more kallikrein polypeptides, in a first sample obtained from a patient and exposed to the test agent, wherein the kallikrein polypeptides comprise or are selected from the group consisting of kallikrein 7 and kallikrein 10, and (b) levels of the kallikrein polypeptides in a second sample obtained from the patient, wherein the sample is not exposed to the test agent, wherein a significant difference in the levels of expression of the kallikrein polypeptides in the first sample, relative to the second sample, is an indication that the test agent is potentially efficacious for inhibiting a neurodegenerative disease in the patient.

15. A method of claim 14 wherein the first and second samples are portions of a single sample obtained from the patient.

16. A method of claim 14 wherein the first and second samples are portions of pooled samples obtained from the patient.

17. A method of assessing the efficacy of a therapy for inhibiting a neurodegenerative disease in a patient, the method comprising comparing: (a) levels of one or more kallikrein polypeptides in a first sample obtained from the patient, wherein the kallikrein polypeptides comprise or are selected from the group consisting of kallikrein 7 and kallikrein 10, and (b) levels of the kallikrein polypeptides in a second sample obtained from the patient following therapy, wherein a significant difference in the levels of expression of the kallikrein polypeptides in the second sample, relative to the first sample, is an indication that the therapy is efficacious for inhibiting a neurodegenerative disease in the patient.

18. A method of selecting an agent for inhibiting a neurodegenerative disease in a patient the method comprising (a) obtaining a sample of cells affected by the disease from the patient; (b) separately exposing aliquots of the sample in the presence of a plurality of test agents; (c) comparing levels of one or more kallikrein polypeptides in each of the aliquots, wherein the kallikrein polypeptides comprise or are selected from the group consisting of kallikrein 7 and kallikrein 13; and (d) selecting one of the test agents which alters the levels of kallikrein polypeptides in the aliquot containing that test agent, relative to other test agents.

19. A method of inhibiting a neurodegenerative disease in a patient, the method comprising (a) obtaining a sample comprising cells affected by the disease from the patient; (b) separately maintaining aliquots of the sample in the presence of a plurality of test agents; (c) comparing levels of one or more kallikrein polypeptides in each of the aliquots, wherein the kallikrein polypeptides comprise or are selected from the group consisting of kallikrein 7 and kallikrein 10; and (d) administering to the patient at least one of the test agents which alters the levels of kallikrein polypeptides in the aliquot containing that test agent, relative to other test agents.

20. A method of assessing the potential of a test compound to contribute to a neurodegenerative disease, the method comprising: (a) maintaining separate aliquots of cells affected by the disease in the presence and absence of the test compound; and (b) comparing expression of one or more kallikrein polypeptides, in each of the aliquots, wherein the kallikrein polypeptides comprise or are selected from the group consisting of kallikrein 7 and kallikrein 10, and wherein a significant difference in levels of kallikrein polypeptides in the aliquot maintained in the presence of the test compound, relative to the aliquot maintained in the absence of the test compound, is an indication that the test compound possesses potential compound to contribute to a neurodegenerative disease.

21. A method of inhibiting a neurodegenerative disease in a patient at risk for developing the disease, the method comprising inhibiting expression of genes corresponding to one or more kallikrein polypeptides, wherein the kallikrein polypeptides comprise or are selected from the group consisting of kallikrein 7 and kallikrein 10.

22. A method for imaging a tissue affected by a neurodegenerative disease comprising:
(a) injecting a tissue affected by a neurodegenerative disease with agents that bind to kallikrein polypeptides, the agents carrying labels for imaging a tissue, and wherein the kallikrein polypeptides comprise or are selected from the group consisting of kallikrein 7 and kallikrein 10;
(b) allowing the agents to incubate and bind to the kallikrein polypeptides; and
(c) detecting the presence of the labels localized to the tissue.

23. A method of claim 1 further comprising an additional kallikrein polypeptide comprising or selected from the group consisting of kallikrein 6 and kallikrein 11.

24. A method of claim 1 wherein the patient sample comprises serum or CSF obtained from the patient.

25. A kit for carrying out a method as claimed in claim 1.

26. A kit for assessing whether a patient is afflicted with a neurodegenerative disease, the kit comprising reagents that specifically bind with one or more kallikrein polypeptides, wherein the kallikrein polypeptides comprise or are selected from the group consisting of kallikrein 7 and kallikrein 10.

27. A kit for assessing the suitability of each of a plurality of agents for inhibiting a neurodegenerative disease in a patient, the kit comprising: (a) the plurality of agents; and (b) reagents for detecting kallikrein polypeptides, wherein the kallikrein polypeptides comprise or are selected from the group consisting of kallikrein 7 and kallikrein 10.

28. A kit as claimed in claim 26 wherein the reagents are antibodies that specifically bind with protein or protein fragments corresponding to kallikrein polypeptides.
